(19) United States
(12) Patent Application Publication
(10) Pub. No.: US 2016/0201131 A1
(43) Pub. Date: Jul. 14, 2016

(54) Method for Identifying Drug Resistance Related Mutations

(71) Applicants: Yan Wang, San Diego, CA (US); Jianming Xu, Beijing (CN); Chun Gong, Nanjing (CN)

(72) Inventors: Yan Wang, San Diego, CA (US); Jianming Xu, Beijing (CN); Chun Gong, Nanjing (CN)

(21) Appl. No.: 14/879,099

(22) Filed: Oct. 9, 2015

Related U.S. Application Data
(60) Provisional application No. 62/062,830, filed on Oct. 11, 2014.

(51) Int. Cl. C12Q 1/68

(52) U.S. Cl. CPC C12Q 1/6883; C12Q 2600/156

(57) ABSTRACT

The present invention provides a method for detecting low frequency drug resistant mutations using ultra deep amplicon sequencing. Multiple selection criteria based on biological relevance were applied to filter out low frequency background noise and lead to reliable identification of drug resistance related mutations. A method to predict the occurrence of drug resistance during the treatment is also provided.

### CROSS-REFERENCE TO RELATED APPLICATIONS

This application claims the benefit of U.S. provisional patent application No. 62/062,830, filed Oct. 11, 2014, the content of which is incorporated by reference herein.

### FIELD OF THE INVENTION

This invention relates to methods and technologies in the field of next generation sequencing, especially related to methods for using next generation sequencing techniques to monitor disease progression and identify drug resistance-related mutations.

### BACKGROUND OF THE INVENTION

Drug resistance (e.g. antibiotics resistance, cancer drug resistance) has become one of most pressing clinical and public health problems. Genetic mutations, either randomly or selectively occurred, in a small subset of clonally expanded cells are at least a partial reason underlying drug resistance in treating diseases like cancer. Identifying the genetic mutations associated with drug resistance is a very important step towards better diagnosis, evaluation of prognosis and treatment of the disease. One of major difficulties of finding these drug resistance associated mutations is the low frequency occurrence of these alleles. Another difficulty to study drug resistance associated mutations lies at obtaining tumor tissue biopsies at various stages of cancer progression.

The next generation sequencing provides very high throughput sequencing capability that enables biomedical researchers to perform experiments at whole exome, whole transcriptome, and even whole genome scale. The high throughput sequencing technology also provides unprecedentedly high resolution into determining individual DNA/RNA sequences of a large population DNAs coming from different sources, allowing detection of low frequency genetic alterations in a population. For example, ultra deep amplicon sequencing can provide more than 10,000× coverage at one single nucleotide position, which enables not only making binary present/absent calls, but also determining the variant frequency at each position. It is therefore suitable for using to detect low frequency genetic alterations that underlie the evolution of drug resistance. One of the major challenges in low frequency mutation analysis is to differentiate the authentic genetic mutations from random errors inherent to the sequencing technique itself or coming from other random sources.

As such, there is a great need for developing technologies to use next generation sequencing techniques to identify drug resistance related mutations as well as to monitor genetic alterations during the progress of a disease for better prognosis. The present invention satisfies this need and provides other benefits as well.

### SUMMARY OF THE INVENTION

The present invention provides a method of analysis of the low frequency mutations in cell-free circulating DNA extracted from patient blood samples to monitor the occurrence of single nucleotide mutations during the treatment of a disease and identify the single nucleotide mutations associated with drug resistance. The amplicon sequencing can provide ultra deep covering of targeted genes that are members of signal transduction pathways related to drug actions. It is used to detect the occurrence of low frequency alleles present only during the progression of the disease and those alleles whose frequencies are increased longitudinally with the treatment of the drug. These mutations are most likely to correlate to drug resistance developed along the drug treatment.

In one embodiment, the present invention provides a method of identifying a drug resistance-related mutation, comprising comparing variant frequencies of a single nucleotide mutation in genomic DNA from a patient before drug treatment and at different time points during the drug treatment, wherein the single nucleotide mutation with increased variant frequency during drug treatment is the drug resistance-related mutation.

In one embodiment, the variant frequency of the single nucleotide mutation is determined by amplicon ultra deep sequencing.

In one embodiment, the DNA sequence is isolated and amplified from the genomic DNA extracted from cell free circulating DNA in plasma.

In one embodiment, the drug resistance-related single nucleotide mutation is detected by comparing single nucleotide mutations in pretreatment DNA samples vs. DNA samples obtained at various stages of drug treatment or at the progression stage of the disease.

In one embodiment, the drug resistance-related mutations are only present in DNA samples after the drug treatment, and do not exist in the pre-treatment DNA samples. In another embodiment, the drug resistance-related mutation has increasingly higher variant frequency in longitudinal DNA samples during the course of drug treatment.

In one embodiment, the present invention provides a method of identifying drug resistance-related mutations in a patient, comprising the following steps: a) calculating variant frequencies of single nucleotide mutations in DNA samples of the patient at pre-treatment and disease progression stage; b) identifying single nucleotide mutations with significantly higher variant frequency in DNA samples at progressive disease (PD) stage than that at pretreatment stage and collecting these single nucleotide mutations to make a PD-preferred mutation group; c) selecting outlier mutations in the PD-preferred mutation group that have significantly higher variant frequencies at the PD stage than those of the rest mutations in the same group, wherein the single nucleotide mutations with high variant frequency outliers are drug resistance-related mutations.

In another embodiment, the present method further comprises selecting single nucleotide mutations with variant frequency increasing in longitudinal samples during the course of drug treatment, wherein the selected mutations are the drug resistance-related mutations.

In another embodiment, the present method further comprises selecting single nucleotide mutations with gain or loss of function effect on a protein.

In another embodiment, the present invention provides a method of predicting the occurrence of drug resistance, comprising the steps of: a) calculating variant frequencies of single nucleotide mutations in DNA samples of a patient at pre-treatment stage and different time points during the course of drug treatment; b) collecting a pool of mutations (treatment-preferred mutations) wherein the variant frequencies are significantly higher in the drug treatment DNA samples than those in pretreatment samples; c) detecting the occurrence of high frequency outlier mutations in the treatment-preferred mutation pool, wherein the occurrence of the high frequency outlier mutation predicts the coming occurrence of drug resistance.

In one embodiment, the variant frequency used for prediction of drug resistance is determined by amplicon ultra deep sequencing.

In one embodiment, the DNA sample used for calculating variant frequency is cell free circulating DNA in patient's blood sample.

In another embodiment, the treatment-preferred mutations are only present in the DNA samples after drug treatment, and are absent in the pretreatment DNA sample.

### BRIEF DESCRIPTION OF DRAWINGS

FIG. 1. Patient enrollment and clinical sample collection. Using targeted amplicon deep sequencing, 25 candidate mutations were identified from 32 patients with acquired Cetuximab resistance by comparing mutation profiles of plasma from progression and before treatment. Those mutations are not present in 12 patients with primary resistance. Pts, patients

FIGS. 2A-2D. Mutation profile in targeted gene panel. FIG. 2A shows distribution of mutations in primary and acquired resistance patients in tumor tissue. Mutations are more evenly distributed among target genes in patients with primary resistance while they cluster in EGFR and TP53 and low in other genes in patients with acquired resistance. FIG. 2B shows mutation profiles detected in tumor tissue and pre-treatment plasma from both primary and acquired resistant patients. 39 mutations were identified in 38 acquired resistant patients while 23 mutations were identified in 12 primary resistant patients, averaging 1 and 2 mutations per individual respectively. Some patients had multiple mutations as exhibited in different colored blocks. A fraction of mutations in tumor tissues are also detected in plasma. Those patients tend to have shorter PFS. FIG. 2C shows the distribution of mutations screened to be related to acquired resistance in ctDNA from individual patients grouped by genes. Single and double mutations in one gene are highlighted in different colors. FIG. 2D shows percentage of mutations in each gene. The gene harbored the most mutations was KRAS with PIK3CA came in second.

FIGS. 3A-3B. Comparison of circulating mutant DNA and tumor biomarkers to monitor tumor dynamics and acquired resistance to Cetuximab. FIGS. 3A-3B show mutation frequencies in serial circulating tumor DNA, CEA (ng/ml) and CA19-9 (U/ml), and disease status as ascertained on computerized tomography (vertical dotted lines) for four patients (two in FIG. 3A and two in FIG. 3B). Longitudinal samples start with before treatment and end at progression. FIGS. 3A-3B showed detectable levels of mutations before progression was determined clinically.

FIGS. 4A-4D. Activation of AKT and ERK1/2 by PIK3CA mutations and sensitivity of Difi cells transfected with PIK3CA variants to cetuximab and 5-FU. FIG. 4A, the mutations (E542K, E545K, H1047K) shown in green color are mutations reported in the literature. The newly discovered mutations are shown in Blue-white color. As shown in the picture, three residues (K944, V955, K966) are located on the surface of PIK3CA protein, H1047 and V952 are half-embedded, and F930 is fully embedded in the protein. FIG. 4B is another graph to show residue distribution of PIK3CA (directly use 4JPS/PDB file, the color scheme is the same as above). It includes PIK3R1 (phosphatidylinostal 3-kinase regulatory subunit alpha) that interacts with PIK3CA. It shows that E542K, E545K and K944N mutations are on the interface of the two protein subunits. FIG. 4C shows activation of AKT and ERK1/2 by PIK3CA mutations. Western blots comparing the phosphorylation levels of AKT and ERK1/2. FIG. 4D shows differential sensitivity of Dif cells transfected with wild type PIK3CA or mutant PIK3CA to cetuxima and 5-FU. Values shown are the mean+/−SD of n=6 experiments. In the legend, cells are ordered in terms of increasing sensitivity to the 10 μg/ml cetuximab and 10 μM 5-Fu concentration.

FIG. 5. Candidate mutations resulted from comparison of plasma before treatment and post progression. Dotted lines and solid line represent mutations frequencies of candidate mutations at progression and before treatment, respectively. Outliers on the dotted line are mutations selected by quartile analysis.

FIG. 6. Workflow of bioinformatics analysis. Bioinformatics analysis workflow consists of three parts: 1. Raw data QC, mapping and SNP calling with no set filters to include mutations with low variant frequencies; 2. Select candidate mutations related to acquired cetuximab resistance in ctDNA, and somatic mutations in FFPE using different strategies; 3. Mutation annotation and protein functional prediction.

FIG. 7A-7G. Comparison of circulating mutant DNA and tumor biomarkers to monitor tumor dynamics and acquired resistance to cetuximab.

FIGS. 8A-8C. Predictive potential of different situations for the progression free survival. FIGS. 8A, 8B and 8C show the Kaplan-Meier survival function of progression free (PFS). All 38 patients with acquired resistance were included in this analysis. Patients with BRAF mutations in FFPF, or with mutations detected in ctDNA before treatment showed significantly shorter PFS as compared with those with wild type BRAF (Harzad Ratio 0.178, 95% confidence interval, 0.054 to 0.585; P=0.004) or without any mutation in ctDNA (Harzad Ratio 0.332, 95% confidence interval, 0.137 to 0.805; P=0.015), respectively. Patients with the presence of residual primary tumor had a meaningful trends towards shorter PFS as compared with those with the absence of residual primary tumor (Harzad Ratio 0.582, 95% confidence interval, 0.268 to 1.262, P=0.170).

FIGS. 9A-9B. Sensitivity of Difi cells transfected with wild type PIK3CA or mutant PIK3CA to cetuximab and 5-FU. FIG. 9A showed differential sensitivity of Difi cells transfected with wild type PIK3CA or mutant PIK3CA to cetuximab. Values shown are the mean+/−SD of n=6 experiments. In the legend, cells are ordered in terms of increasing sensitivity to the 10 μg/ml cetuximab concentration. FIG. 9B discloses differential sensitivity of Difi cells to 48 hours of 5-FU treatment. Values shown are the mean+/−SD of n=6 experiments.

FIG. 10. Distribution of observed non-reference read frequencies in cfDNA. Mutations detected in plasma are mostly low frequency variants. Majority of them are lower than 0.5%. Many low frequency somatic mutations, especially emerging ones, could be buried in the background noise of random errors. Simple filter based on mutation frequencies cannot be used.

FIGS. 11A-B contains “Table 1. Detection of KRAS/PIK3CA and BRAF mutation in tumor tissue and plasma.” *Residue of primary tumor: Y yes, N no. †Source of tumor tissue: P primary tumor, M metastatic lesion. ‡AA change: amino acid change. §AF: mutant allele fraction. ¶ Best response: According to the RECIST criterion, PR partial response, SD stable disease, PD progressive disease.

FIG. 12 contains “Table S1. Clinical Information.” *Residue of primary tumor: Y yes, N no. †Tumor burden: Aggregate cross-sectional diameter of the index lesions. ‡Best response: According to the RECIST criteria, PR partial response, SD stable disease, and PD progression disease. ¶PFS: Progression free survival. **OS: Overall survival

FIGS. 13A-B contain “Table S2. Mutation in tumor tissue and presence in plasma before treatment.” † aa change: amino acid change. ‡ AF: mutant allele fraction. §Source of tumor tissue: P, primary tumor, M, metastatic lesion. ¶ NMD: no mutation genes detected. −: Mutant allele under the detectable level. *: Stop-gain mutation

FIG. 14 contains “Table S3. Functional prediction of mutations detected in FFPE specimens by PolyPhen-2 and SIFT.” Among 65 mutations, 47 mutations determined to be “damaging”, including 33 nonsynoumous mutations selected by both software of PolyPhen-2 and SIFT, 9 stop-gain, 5 known hot spot mutations at KRAS, NRAS and TP53 marked as #.

FIG. 15 contains “Table S4. Functional prediction of mutations detected in circulating tumor DNA by PolyPhen-2 and SIFT.” Among 39 mutations, 19 mutations determined to be “damaging”, including 17 nonsynoumous mutations selected by both PolyPhen-2 and SIFT, one stop-gain and one known hot spot mutation at KRAS Q61H.

FIG. 16 contains “Table S5. Mutations related to acquired resistance in circulating tumor DNA.” †aa change: amino acid change. *: Stop-gain mutation.

FIGS. 17A-B contain “Table S6. Mutations related to acquired resistance in circulating rumor DNA.” † aa change: amino acid change. ‡ AF: mutant allele fraction. §NA: plasma sample not available. ¶ NMD: no mutation genes detected. −: Mutant allele under the detectable level. *: Stop-gain mutation. #: Last plasma sample collected. Patient didn't reach progressive disease.

### DETAILED DESCRIPTION OF THE INVENTION

### Definitions

Unless otherwise defined, all technical and scientific terms used herein have the same meaning as commonly understood by one of the ordinary skill in the art to which this invention belongs.

The term “disease progression” or “progressive disease (PD)” as used interchangeably herein, refers to a time point during the disease treatment when the disease shows more than a slight increase in size or extent on or after treatment. For example, in the treatment of cancer, a cancer drug may initially be able to control or even decrease the size and the number of tumors (Stable Disease (SD) stage). When drug resistance emerges in the course of treatment, the tumors will start to grow in size and number again (PD stage), which can be clinically monitored by imaging techniques like Computerized tomography (CT) scan. The time point when the increase of tumor size and number can be detected by imaging and other empirical techniques is considered the entry of disease progression or progressive disease stage. Progressive disease indicates that the administered drug treatment was not effective and drug resistance is clinically evident. Other treatments may be necessary to control the disease at PD stage. The term “progression free survival (PFS)” refers to the period when the disease is under control by the drug and no further deterioration is found in the patient. PFS period ends when disease progression is clinically detected.

The term “variant frequency” used herein refers to the proportion of a particular variant (allele) among all the allele copies for a genetic locus in a population. It is usually expressed as the percentage of the number of copies of a particular allele divided by the total copy number of all alleles of a genetic locus in a population. The variant frequency used herein especially refers to the percentage of copies of a particular single nucleotide mutation (variant) in all the allele copies of a gene detected in a patient's genomic DNA sample. The change of variant frequency profile reflects the genetic changes in patient's genomic DNA pool during the course of drug treatment. Genomic DNA extracted from cell free circulating DNA in plasma has DNA sequences coming from normal cells as well as tumor cells. There is only small portion of DNA contains alleles specific to tumor cells, whereas there is even smaller portion of DNA will contain alleles that are specifically related to drug resistance. The techniques and methods to examine these low frequency mutations can provide important information to monitor and guide the progress of therapeutic treatment.

The term “outlier” in statistics generally refers to a value that lies at an abnormal distance from other values in a random sample from a population. The term “outlier mutation” used herein refers to a mutation that has an abnormally high variant frequency compared to those of other mutations in the same group. This abnormally high variant frequency of a mutation signals that the mutation may not happen by random chance, but be accumulated by directed selection, for example, under the positive selection pressure of drug resistance. Combined with evaluation of the functional significance of this mutation and study of its dynamic trend during the course of drug treatment, it is possible to make a convincing connection between the outlier mutation and drug resistance. Methods to identify outliers in a population is well known to one of ordinary skills in the art, including, but not limited to, graphic methods such as box plot and scatter plot, and analytic methods such as Grubb's test and Tietjen-Moore Test.

The term “drug resistance-related mutation” used herein refers to a mutation or mutations of a gene that are correlated with drug resistance. The drug resistance-related mutation(s) could be a cause or a consequence in relation to the drug resistance. The variant frequency of a drug resistance-related mutation should increase in a patient's DNA population with the prolonged treatment of the drug due to positive selection pressure. This sets the basic strategy for looking for such mutations.

One of major challenges in clinical studies of drug resistance lies in the difficulties of obtaining multiple tumor biopsies during the treatment process and the biased representation from single tumor biopsies in metastatic cancer patients with multiple solid tumors. The problem of lacking solid tissue biopsy is partially solved by turning into “liquid biopsy” for patient's DNA, where cell free circulating DNA (cfDNA) isolated from patient's plasma can provide an unbiased representation of patient's genetic makeup during the course of drug treatment. The cfDNA in the plasma has DNA coming from all different types of cells in the body including DNAs from normal cells as well as tumor cells. Using amplicon ultra deep sequencing techniques, it is possible to detect low frequency variants in the mixture of all alleles from different cell sources. This brings up another challenge: how to specifically identify authentic low frequency drug resistance-related alleles from the crowd of low frequency variants, most of which come from random noise of sequencing technique itself and other sources. This invention provides a novel multi-step strategy to filter out random noises and reliably screen for drug resistance relevant variants.

The present invention provides a method for monitoring genetic alterations during the disease treatment and identifying drug resistance-related mutations at individual patient level, which is helpful for developing personalized therapeutic strategy tailored to each patient's unique situation. This method is especially useful for diseases like cancer which has a strong genetic component. The invented method is designed to detect low frequency, yet biologically significant, genetic mutations, which could be otherwise missed from the detection radar. The rationale of this invention is to use more than one criteria to filter out random noises and let real signals stand out of the crowd. It is reasoned that a genuine drug resistance-related mutation should be under positive selection and have significantly increased levels during the course of drug treatment. The criteria to look for such mutation include: 1) its variant frequency is significantly increased after treatment vs. pre-treatment; 2) its variant frequency has a longitudinally increasing trend along the treatment process; 3) the mutation is non-synonymous and can lead to loss or gain of function changes; or 4) the mutation only exists after drug treatment, but is not detected in pretreatment samples. Using more than one selection criteria allows starting search in very low frequency (e.g. <0.5%) variant pools, filtering out random low frequency variant noise in a step-wise manner, and finally letting drug resistance-related variants stand out.

In one embodiment, the present invention provides a method of identifying a drug resistance-related mutation by comparing variant frequencies of a single nucleotide mutation in genomic DNA from a patient before drug treatment and at different time points during the drug treatment, and look for the single nucleotide mutation with significantly increased variant frequency during drug treatment, which is likely to be the drug resistance-related mutation.

The DNA sample used in this method should have a good representation of tumor DNA of the patient. For example, the DNA sample may be obtained from tumor tissue biopsy or, preferably, from free circulating DNA in patient's plasma. The fcDNA is preferable because it is easier and inexpensive to obtain patient's plasma sample and the fcDNA, which constitutes the shredded DNA from all cell types, provides an more even presentation of all the tumor DNAs in the body. The genetic alterations in fcDNA can serve as a good barometer for the dynamic change of patient's genetic landscape during the course of drug treatment. Techniques to measure low frequency variants in a DNA population are currently available. For example, ultra deep amplicon sequencing can measure individual DNA sequences in a mixture of DNAs arising from different sources and can have more than 10,000× coverage at each nucleotide location, allowing detection of variants with an occurring frequency less than 0.5%.

To find drug resistance-related mutations, the first step is to make single nucleotide variant (SNV) calling and compare the variant frequencies in DNA samples at pre-treatment stage and a time point after treatment, preferably at disease progression, to construct a pool of variants that have significantly higher frequencies in treatment samples than those in pretreatment samples. This pool of variants is called treatment-preferred mutations. The methods to make SNV calling and calculate variant frequencies are well known to one with ordinary skills in the field (1-4), and software applications for finding somatic mutations in multiple tissue samples of the same individual such as VarScan, SomaticSniper and SomaticCall are commonly available and have been widely used in the field to search tumor-specific somatic mutations by comparing DNAs in tumor tissue and paired normal tissue. In the claimed invention, the same software applications are used to construct a pool of treatment-preferred mutations or PD-preferred mutations by comparing variant frequencies of mutations in treatment samples vs. paired pretreatment samples. The treatment-preferred mutation pool consists of mutations with variant frequencies significantly higher in treatment samples than those in pretreatment samples. The PD-preferred mutation pool consists of mutations that only exist at PD stage, but are not detectable in pretreatment samples.

The next step is to compare the variant frequencies at treatment stage/PD stage and look for outliers of high variant frequency in the pool of treatment-preferred mutations or PD-preferred mutations. If a high frequency outlier is found in the treatment-preferred or PD-preferred pool, it is very likely that the high frequency outlier mutation is a drug resistance-related mutation. The detection of an outlier mutation can indicate either a bad data point or a significant event that is driven by a different underlying forces, e.g. under positive selective pressure of drug resistance. To differentiate these two possibilities, the third and fourth criteria can come into play. If the longitudinal studies show that the outlier mutation has an increasing frequency trend along the course of drug treatment and/or the mutation can cause gain or loss of function effect on key proteins, these consistent observations will provide strong support that the outlier mutation is not a random error, but a likely drug resistance mutation. In the examples described below, we found that most outlier mutations also consistently exhibit variant frequency uptrend during the treatment and are likely to locate at positions important for protein function. In addition, the occurrence of a high frequency outlier have a high chance of predicting the advent of disease progression with an average of 10 weeks lead time (4.0-18.1 weeks). By detecting the occurrence of high frequency outlier mutation at different time points during the treatment, it holds a great promise for predicting the emergence of drug resistance. If there is no high frequency outlier detected in the treatment-preferred group or PD-preferred group, the mutation having the highest variant frequency can be a drug-resistance candidate. This candidate will be subjected to longitudinal test and functional test to see if it has positive outcomes in both tests. If the candidate shows a clear uptrend in variant frequency during the course of the treatment, and it has loss or gain of function effect on key proteins, it is still reasonable to believe that this candidate is likely to be a drug resistance-related mutation.

In another embodiment, the present invention provides a method of predicting the occurrence of drug resistance, comprising the steps of: a) calculating variant frequencies of single nucleotide mutations in DNA samples of a patient at pre-treatment stage and different time points during the course of drug treatment; b) collecting a pool of mutations (treatment-preferred mutations) wherein the variant frequencies are significantly higher in drug treatment samples than those in pretreatment samples; c) detecting the occurrence of high frequency outlier mutations in the treatment-preferred mutation pool, wherein the occurrence of the high frequency outlier mutation predicts the future occurrence of drug resistance.

As explained above, the occurrence of a high frequency outlier mutation often foresees the coming drug resistance that later become detectable by clinical tests or imaging techniques. This is not surprising because the existence of a high frequency outlier drug resistant mutation indicates that the drug resistant mutation has accumulated to such a high degree that it will soon manifest into phenotypic changes in the patient, that is, disease progression. Therefore, detection of the high frequency outlier mutation is a powerful tool to predict the treatment resistance and provide guidance to prescribe alternative therapy.

### Examples

The following examples are provided for illustration purposes, are not intended to limit the scope of the invention, which is limited only by the claims.

### Patients and Sample Collection

We carried out a prospective, single-center study to monitor the dynamic changes of tumor related mutations during disease progression (PD) in cell free circulating DNA, thus to identify mutations that correlate and/or cause acquired resistance to cetuximab in patients with metastatic colon cancer. The study was approved by the research ethics committee of the Affiliated Hospital, Academy of Military Medical Sciences, China.

Eligible patients were histologically confirmed colorectal adenocarcinoma with metastatic disease, and determined to be wild type in KRAS codons 12, 13, 61 and BRAF codon 600 by Sanger Sequencing of DNA extracted from tumor biopsies. Tumor biopsies and blood samples were collected before therapy to be used as reference for somatic mutations. Longitudinal plasma samples (2 ml each) were collected from each patient starting before therapy followed by sampling every 4 weeks during therapy until PD to monitor changes in tumor related mutation profiles. Informed consent was obtained from each patient.

CEA and CA 19-9 were done every therapeutic cycle (2-3 weeks). Computerized tomography (CT) was performed and reviewed in a blinded fashion every 6 weeks to evaluate clinical response based on Response Evaluation Criteria in Solid Tumor (RECIST), version 1.1.

### Sample Processing and Amplicon Sequencing

For mutational analysis of various clinical samples, DNA was extracted from specimen, quantitated and subject to amplicon deep sequencing of the following target genes: NRAS (exon 2, 3 and 4), KRAS (exon 2, 3 and 4), BRAF (exon 15), PIK3CA (exon 19), AKT (exon 3), TP53 (exon 5, 6 and 7), PTEN (exon 5, 7 and 8), and EGFR (exon 10 and 12). Resulting amplicon libraries are subject to deep sequencing on the Proton System (Lifetech, Inc.). Primer sequences and detailed experimental methods were explained in the Supplementary Appendix.

### Identification of Acquired Resistance Mutations

Deep sequencing data processing and single nucleotide variants (SNVs) calling are described in Supplemental Appendix. The methods to identify SNVs in next generation sequencing data are well known to one with ordinary skills in the field. Somatic mutations were identified by comparing SNVs of genomic DNA extracted from paired peripheral blood mononuclear cells (PBMCs) and FFPE samples. Candidate mutations associated with acquired drug resistance were identified by the following steps: 1. Use Varscan to identify somatic mutations present only in plasma obtained at disease progression by comparing SNVs called in cfDNA samples from disease progression to pre-treatment; 2. Order the value of mutation frequencies (low to high) by quartile and obtain the quartile values (Q1, Q2, Q3). Calculate inter-quartile range (Qd=Q3−Q1). All mutation frequencies (M_{f}) are then scanned by the following expression: M_{f}−Q3>1.5 Qd. Mutations satisfying the criteria will enter next step; and 3. Select mutations with variant frequencies increasing in longitudinal samples. Step 3 was omitted from the procedure for patients with no longitudinal samples. For patients who did not reach PD at the time of analysis, we used the last plasma samples collected as plasma samples at progression.

Mutations identified were annotated using SNPnexus. All Non-synonymous mutations were further analyzed using PolyPhen Grid Gateway and SIFT to determine their potential effect on protein function.

### Functional Analysis of PIK3CA Mutations Identified

### Structure Modeling and Analysis

The MODELLER (version 9v6) software was used for homology modeling. Three-dimensional structures of wild and mutant PIK3CA, binding to PIK3R1, were modeled using the three-dimensional structure of 4JPS in the PDB database as a template. Hydrogen atoms were added using CHARMM (version c32b2) software. The protonation states of titratable residues were determined by an in-house CHARMM script. VMD (version 1.9.1) software was used to view and analyze the modeled structures.

### In Vitro Functional Assays

PIK3CA point mutations K944N, F930S, V955G, V955I, K966E and V952A, L938* were introduced into the full length PIK3CA coding sequence with site-directed mutagenesis and inserted into a expression vector (RC213112, Origene). Difi cells were transfected and subject to cell growth and western blot analyses to determine the changes in phosphorylation levels of AKT1 and other downstream targets. Hot spot mutations in PIK3CA exon 9 and 20 (E542K, E545K and H1047R) were included for comparison. Details of cell culture and transfection conditions are described in detail in Supplementary Appendix.

### Statistical Analysis

Kaplan-Meier methods were used to estimate progression-free survival (PFS). A univariate Cox regression analysis was performed for each of the three variables of interest: BRAF mutation in FFPE, mutation in ctDNA before treatment and residual primary tumor. Hazard ratio (HR), 95% confidence intervals (95% CI) and Wald statistic P values were reported for each model. All statistical tests were performed using SPSS statistics software, version 20.0 (IBM). A P value less than 0.05 was considered statistically significant, HR less than 0.5 was considered as clinical meaningful trends due to the small sample size.

### Patient Information

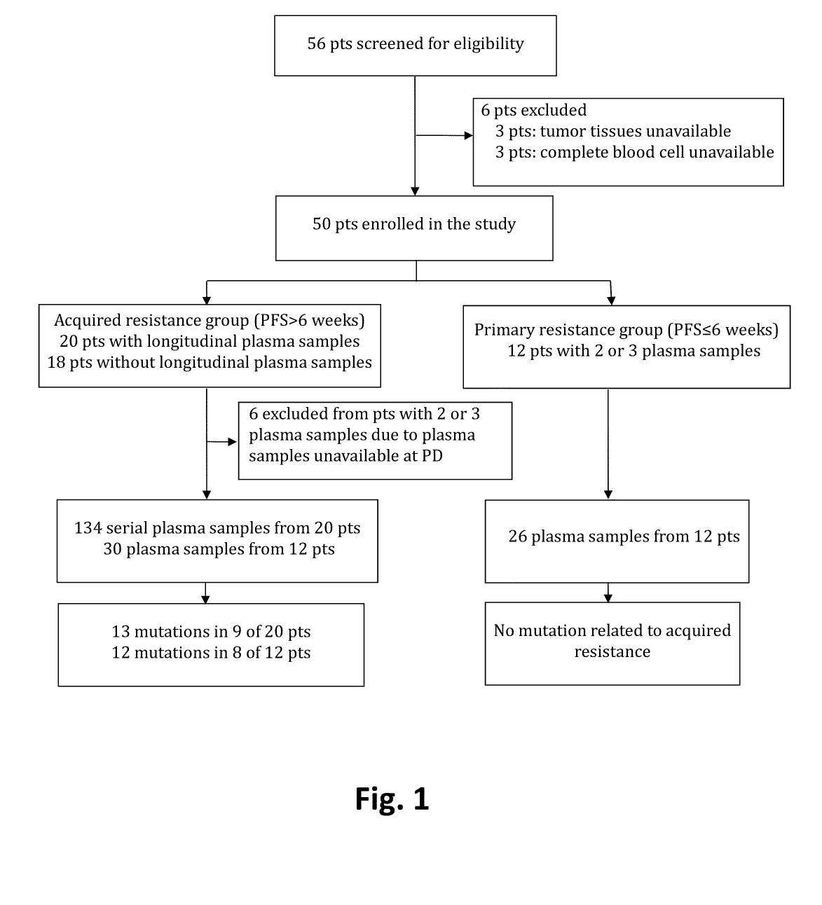

From July 2012 through December 2013. A total of 56 patients were screened. Six were excluded from study due to insufficient collection of tumor or whole blood samples before therapy. Remaining 50 patients have completed at least 6 weeks treatment of cetuximab. Patients were grouped to primary and acquired drug resistance based on results from that first evaluation at week 6: Progression free survival (PFS)≦6 weeks were defined as primary drug resistance while >6 weeks as acquired drug resistance. Among those 50 patients, 38 were categorized as acquired drug resistance group and the rest (12 patients) as primary drug resistance. Clinical information was exhibited in Supplementary Table 1 (Table S1). From 20 out of 38 acquired drug resistance patients, we collected longitudinal plasma samples every 4 weeks, resulting in a total of 134 plasma samples. From the remaining 18 patients, 2-3 plasma samples were collected from each patient. Twelve of those at least had plasma from both before treatment and after PD, resulting in a total of 30 plasma sample, and the rest 6 patients were further excluded from the study due to plasma sample unavailable at progression. We have also collected a total of 26 plasma samples from 12 patients in the primary drug resistance group before and after treatment. In all, our analyses of cetuximab resistance mutations were based on 191 plasma samples from 44 patients (FIG. 1).

### Identifying Somatic Variants in Circulating Tumor DNA

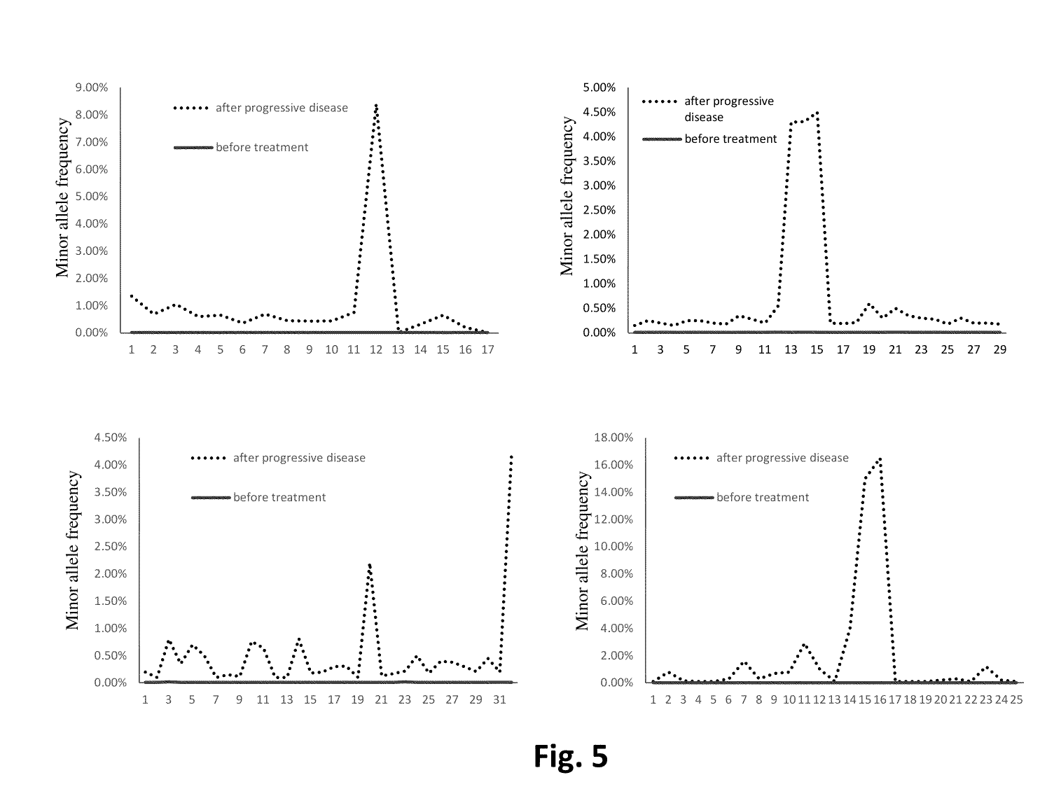

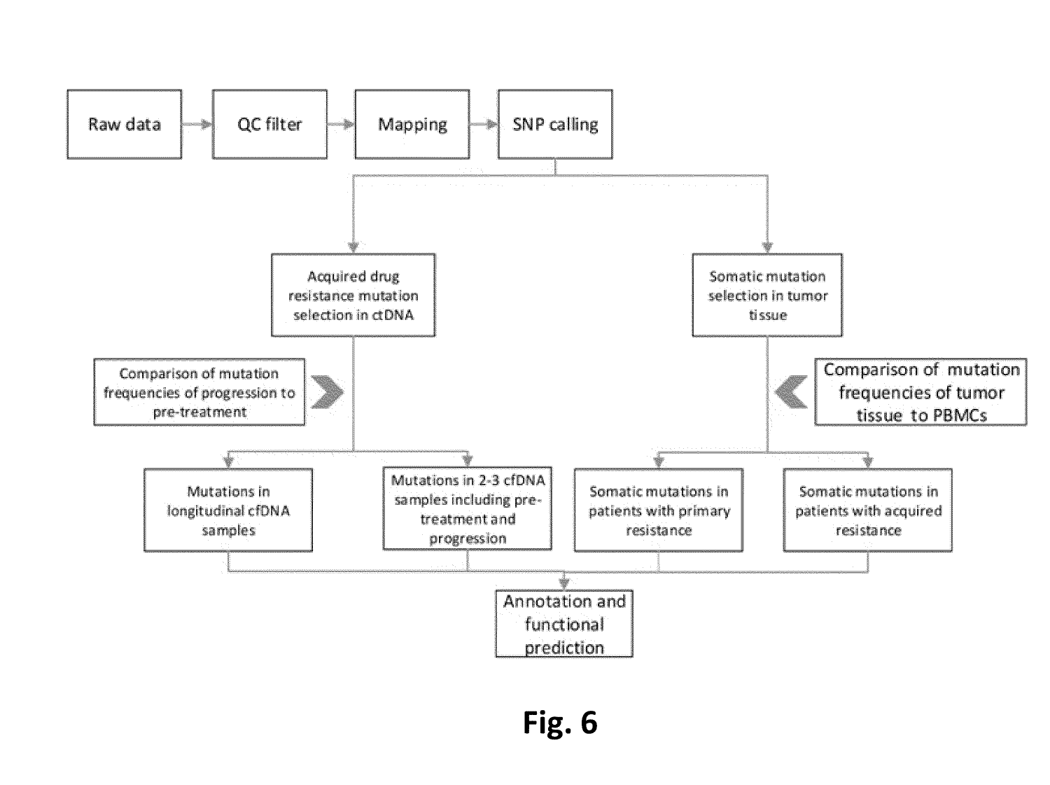

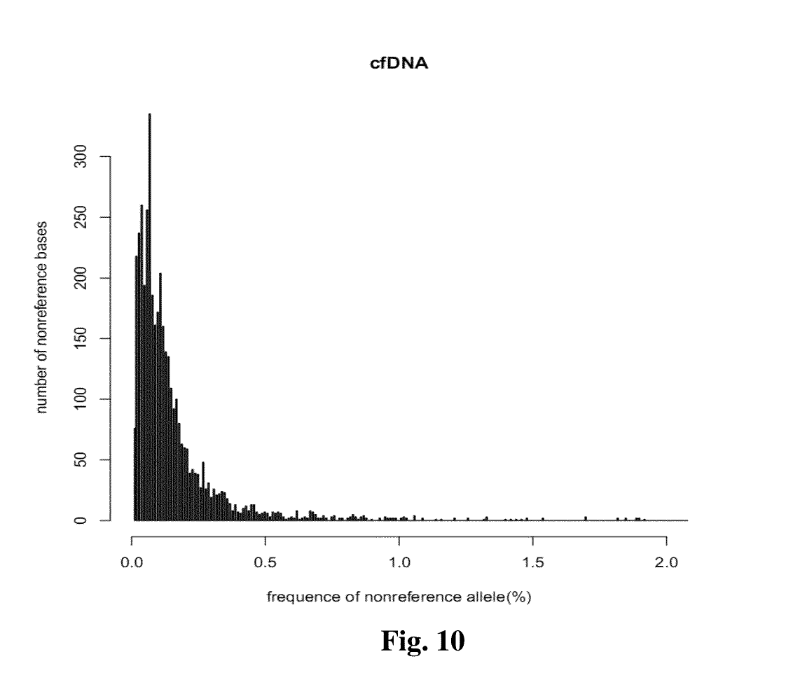

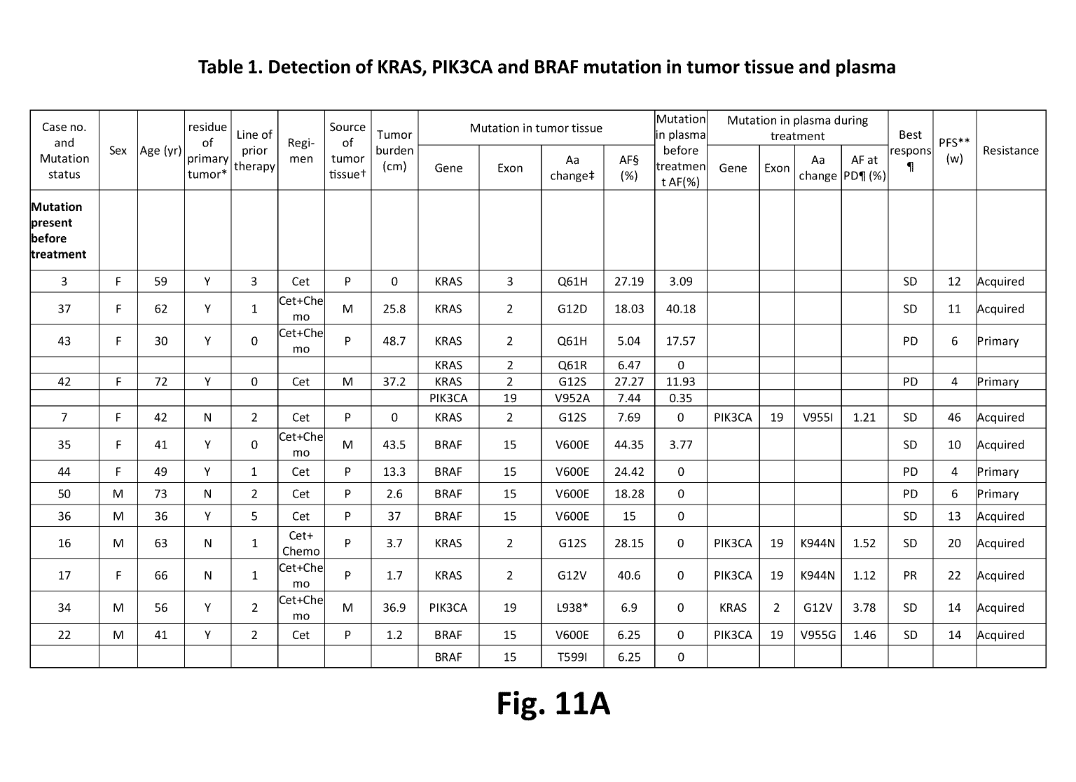

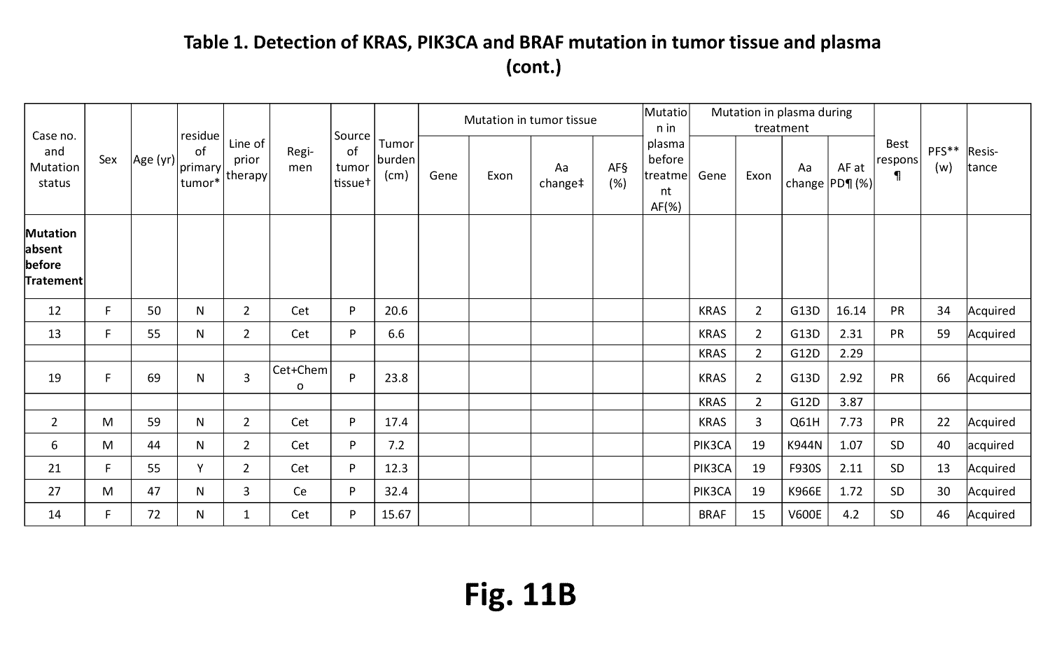

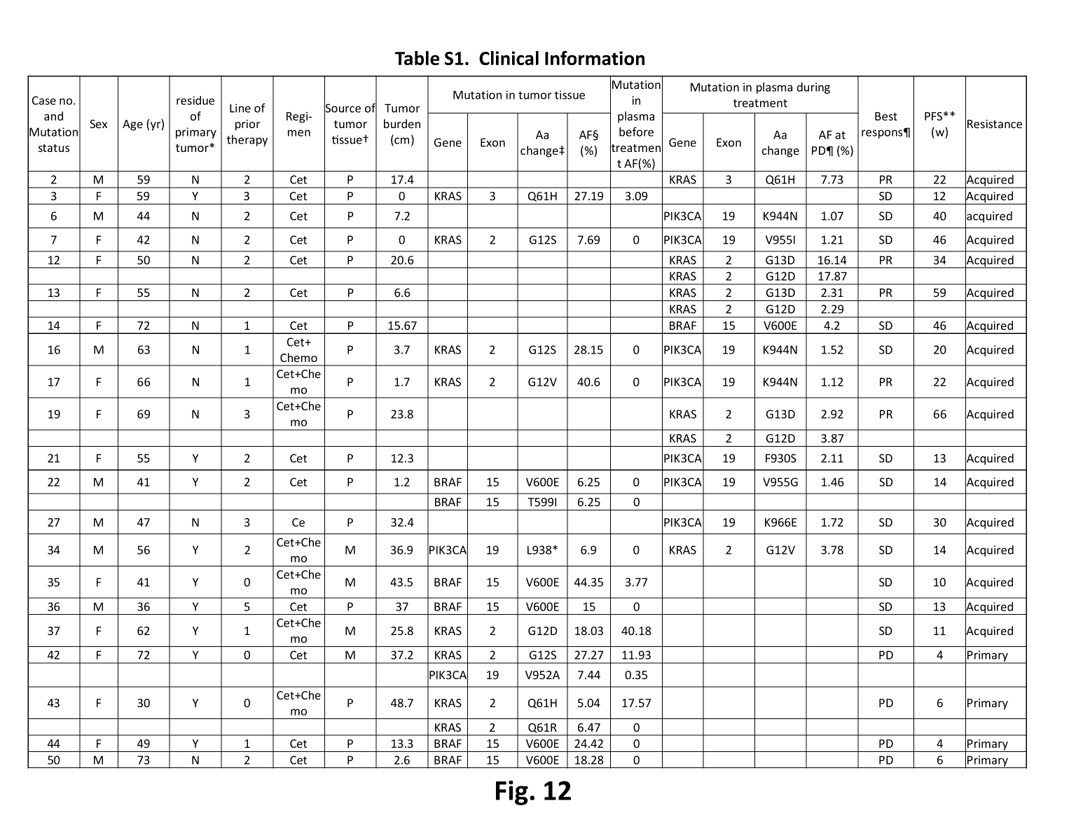

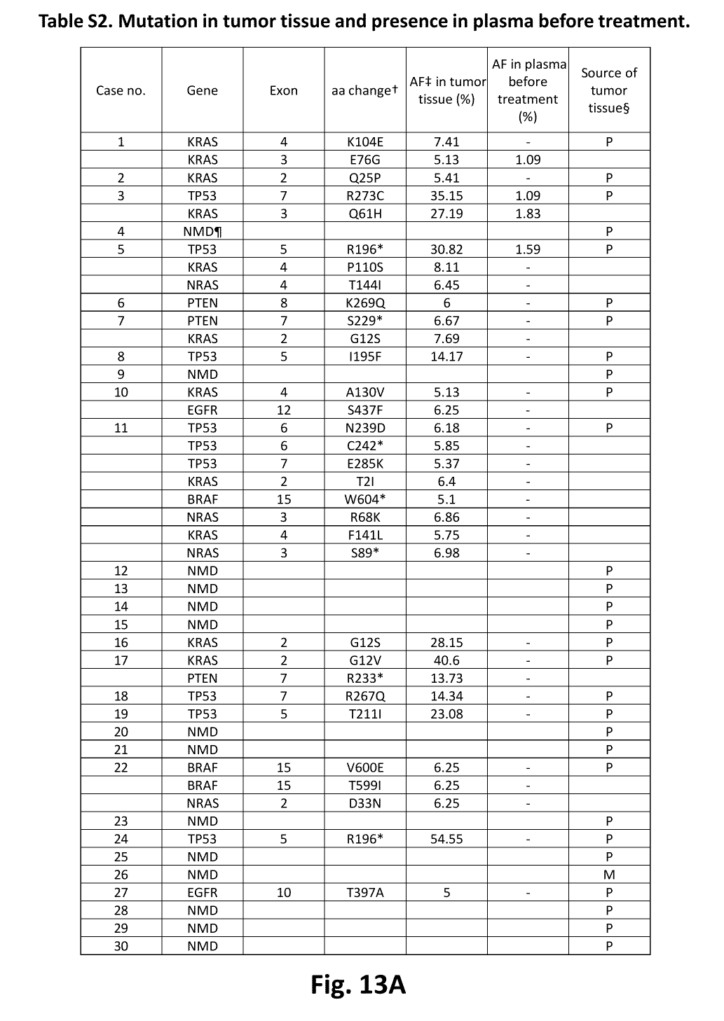

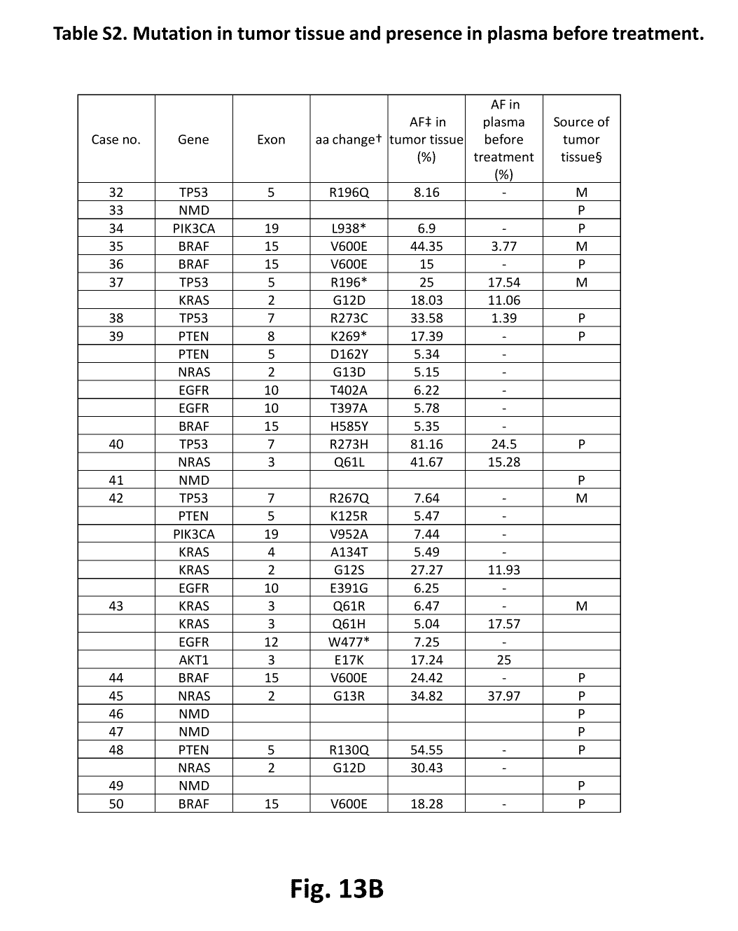

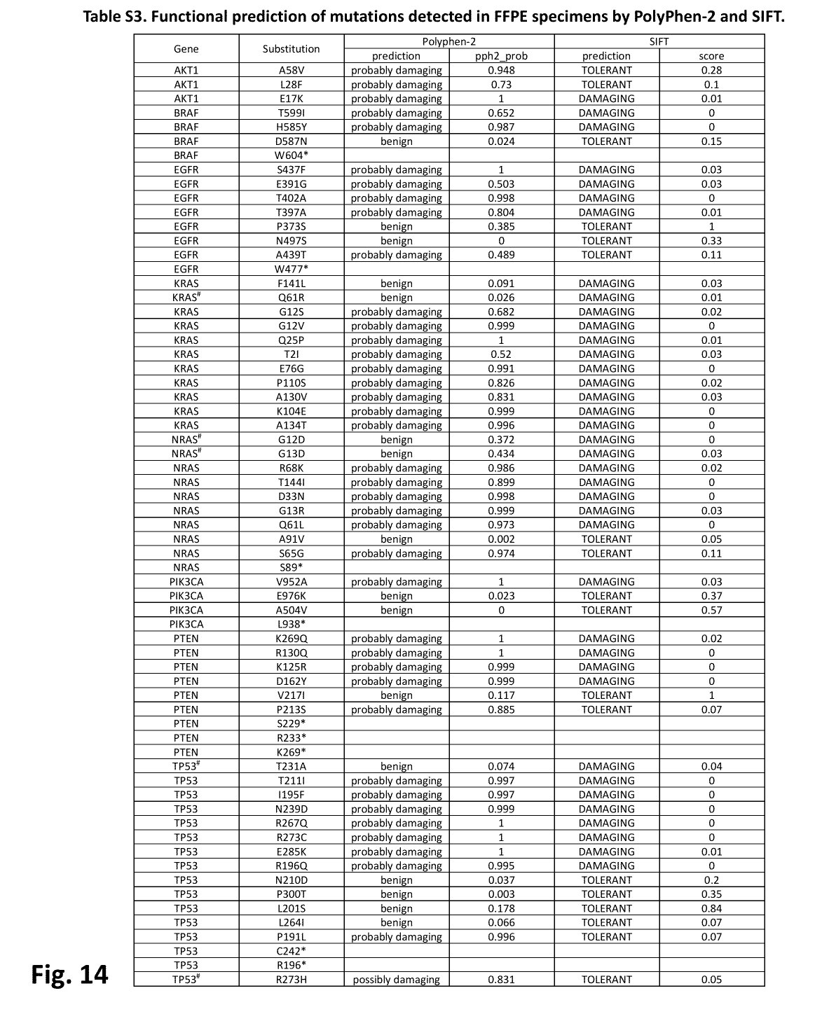

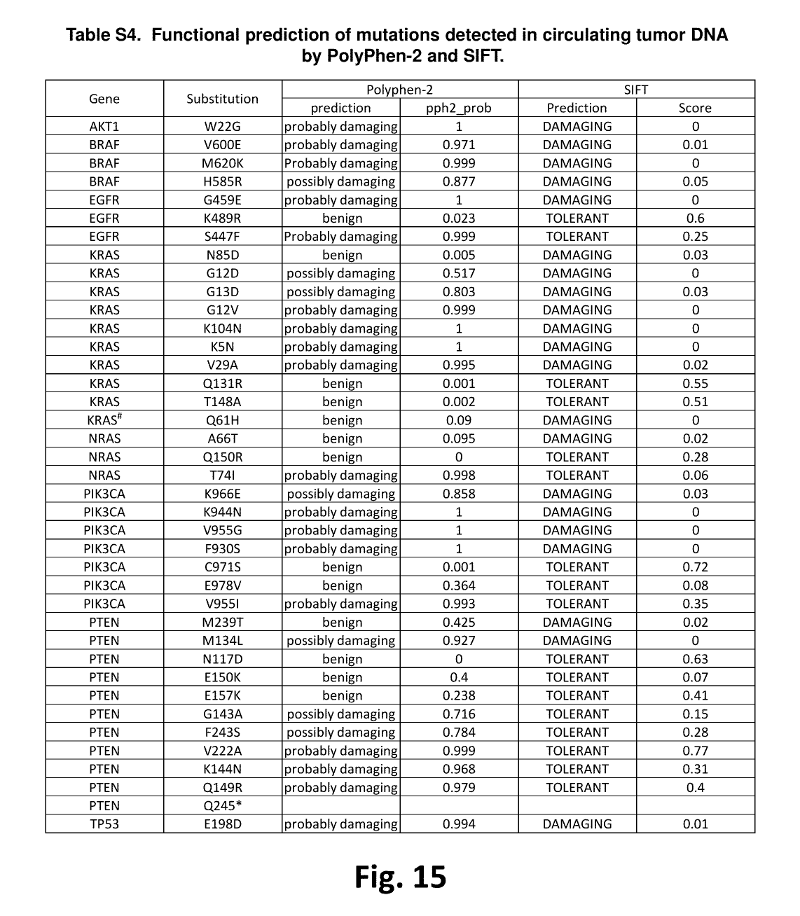

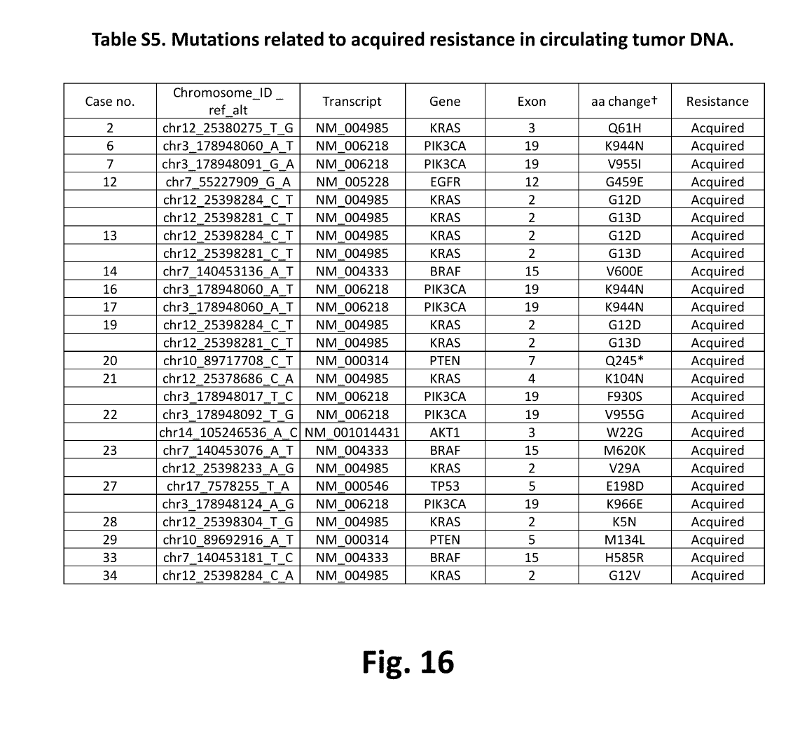

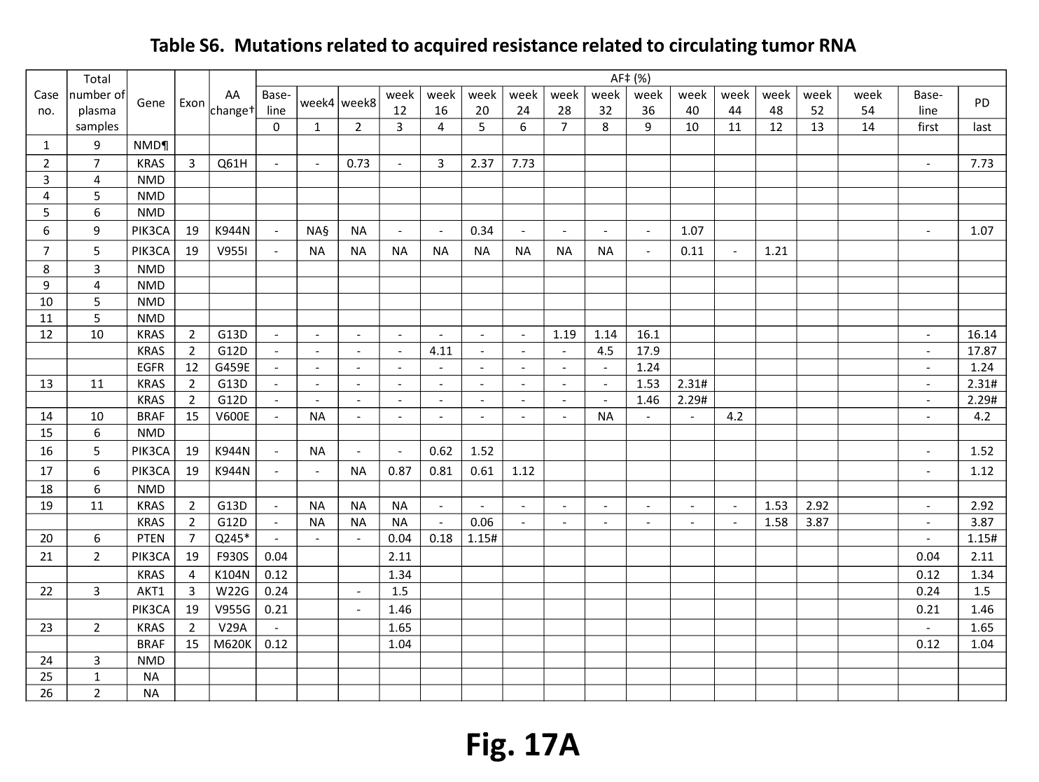

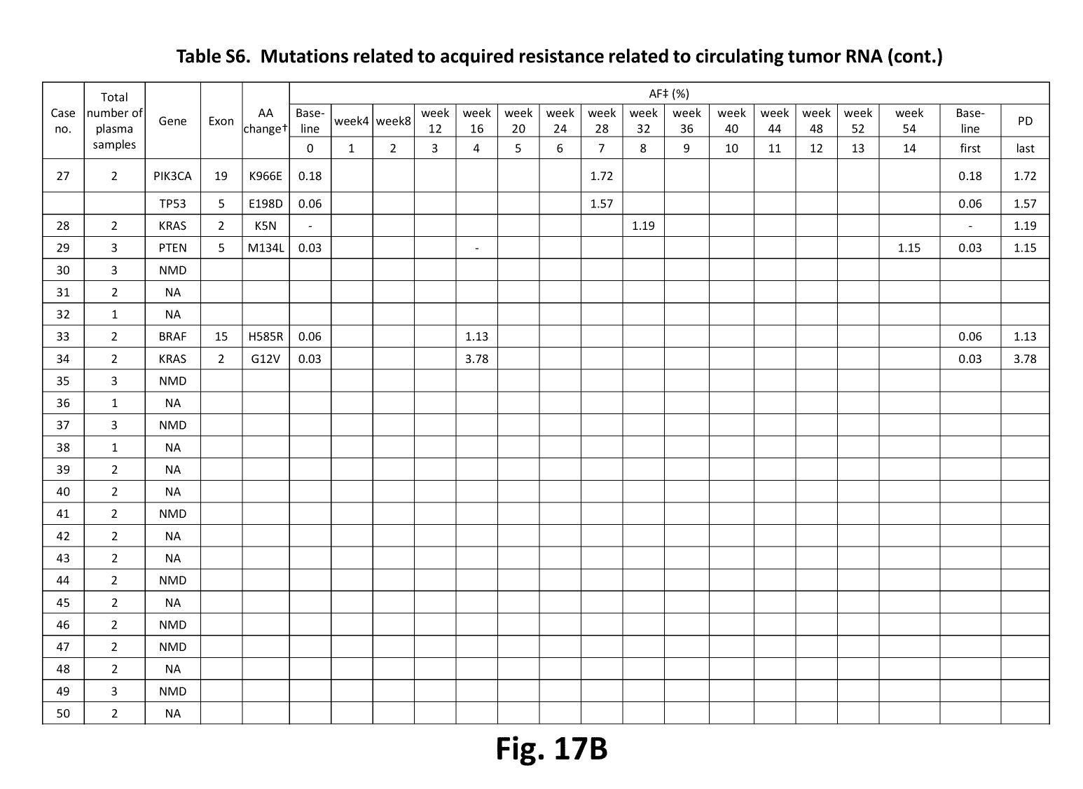

Next, we amplified potential candidate resistant genes of EGFR targeted therapy from ctDNA and performed amplicon sequencing to identify novel mutations that correlate with the emergence of treatment resistance. The average sequencing depth (raw data) for all amplicons in all patients reached 9664×. As expected, majority of the mutations detected had very low variant frequencies of less than 0.5% (FIG. 10). We intentionally called SNVs without filtering for variant frequencies to facilitate future screening. For each patient with longitudinal plasma samples, we first compared mutation profiles from circulating DNA samples collected before treatment and after progression. We identified an average of 26 mutations per person in 11 samples at PD with variant frequencies ranging from 0.1% to 17.87%. The mutations identified for each person were subject to quartile analysis to select outlier mutations with significantly higher variant frequencies than those of the rest mutations for the same person (FIG. 5). We selected a total of 16 mutations in 11 patients, averaging 1.5 per patient. The lowest mutation frequency among the mutations selected was 0.97%. The majority of those mutations showed an increase in variant frequencies in longitudinal plasma samples collected during the treatment. The resultant 16 mutations were considered candidates for acquired resistance of cetuximab. Unlike traditional data analysis approaches for next generation sequencing, such selection strategy helped us identify mutations starting at extremely low frequencies but increase in levels during the treatment.

Using such strategy, we identified 48 non-synonymous point mutations to be candidates for acquired cetuximab resistance from 38 patients including ones with no longitudinal plasma samples and/or did not reach PD at the time of the analysis. These mutations were further screened through two different protein structural functional prediction databases PolyPhen-2 and SIFT. Only those predicted to be “damaging” in both databases were further analyzed in this study (Supplementary Table 3 and 4).

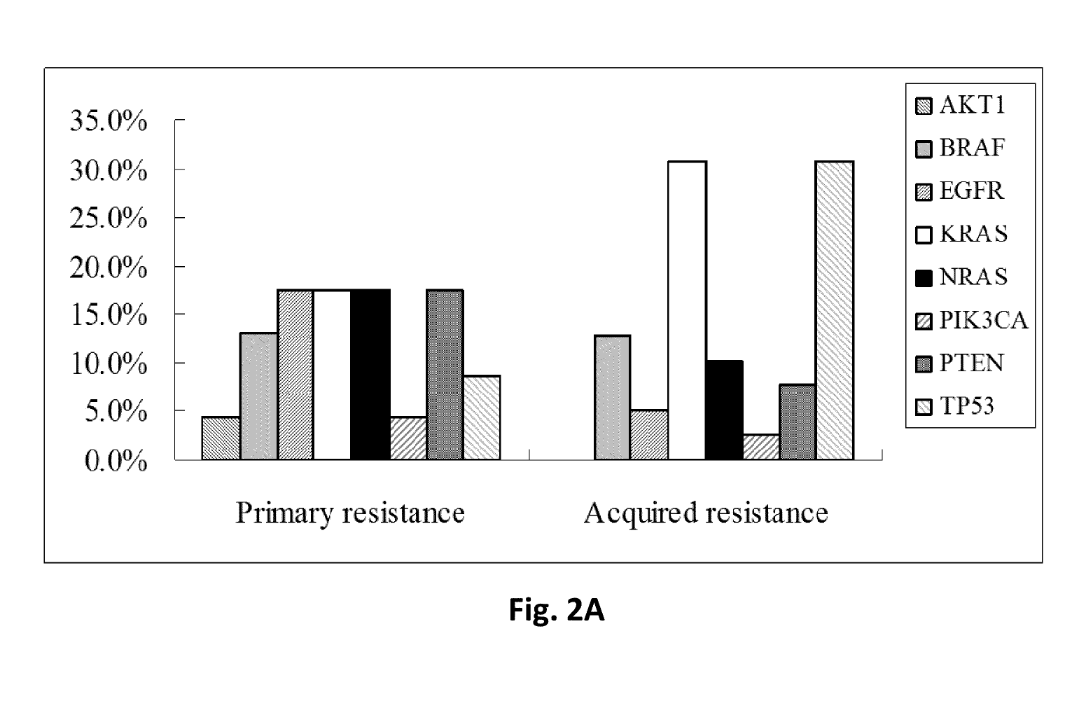

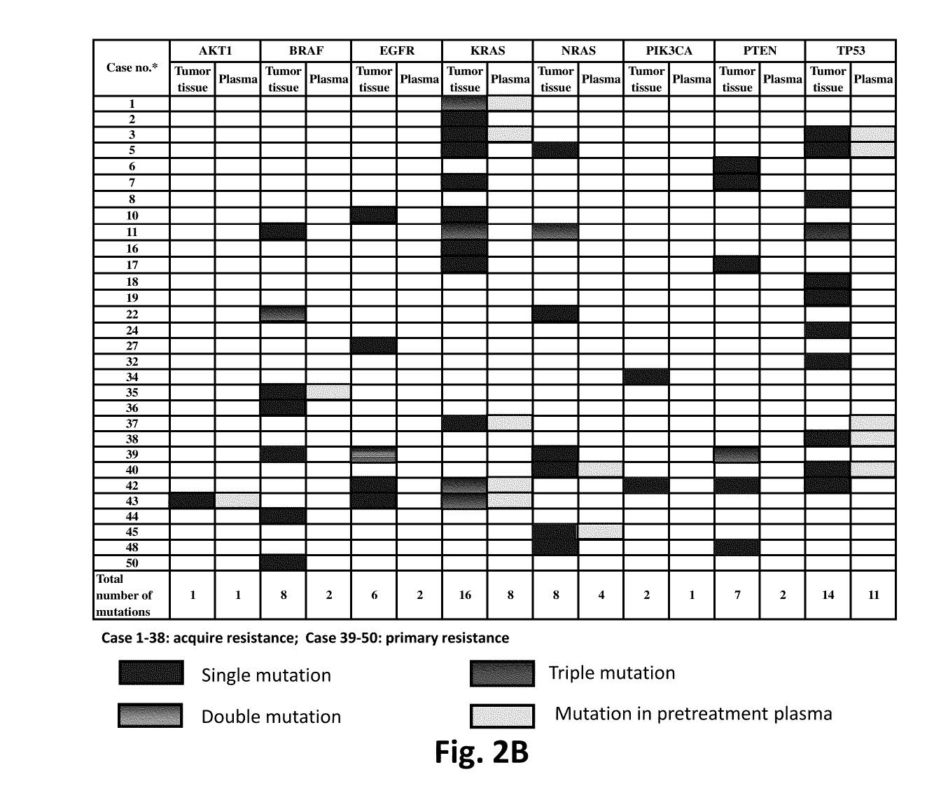

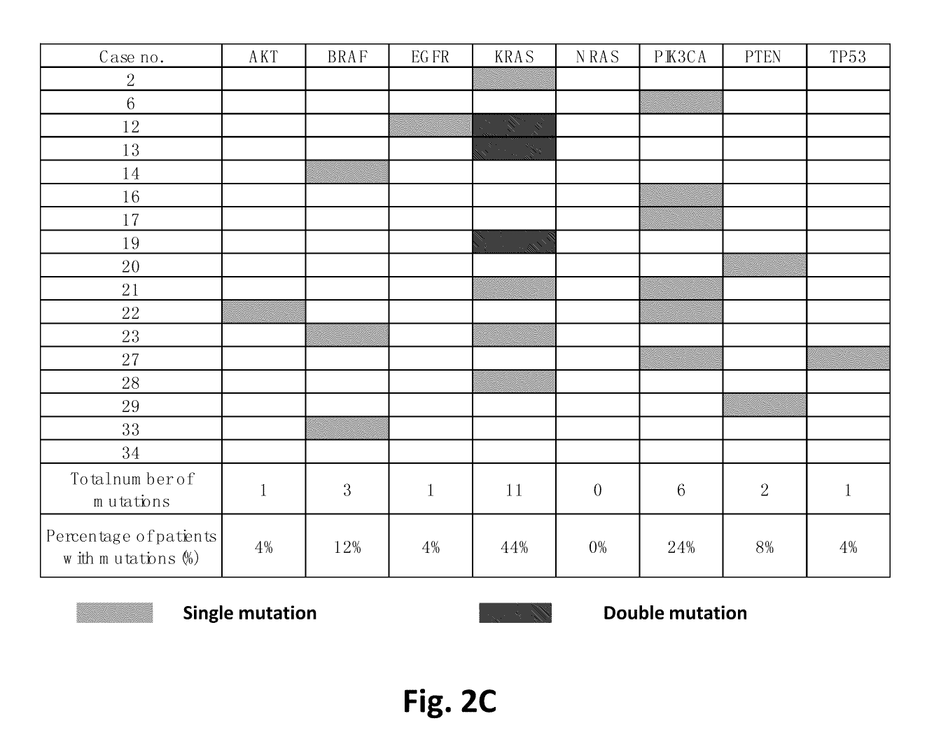

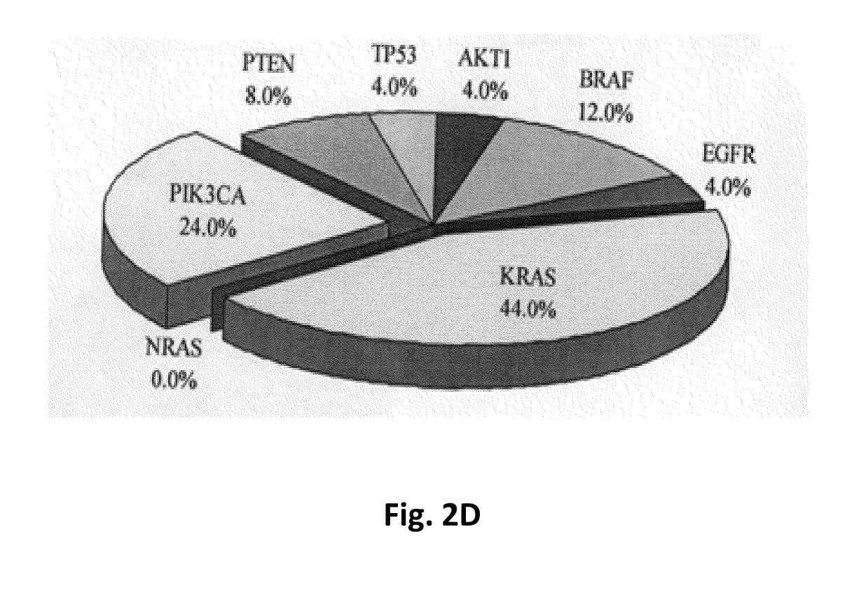

These analyses led to 13 candidate mutations in 9 out of 20 patients with longitudinal plasma samples and 12 candidate mutations in 8 out of 12 patients with at least 2-3 plasma samples including before treatment and after PD. None of those candidate mutations were found in patients in the primary drug resistance group (FIG. 2c). The results demonstrate that 25 (13+12) mutations identified correlate with acquired resistance to cetuximab, among which KRAS (44%), PIK3CA (24%) and BRAF (12%) are in the majority (FIG. 2d). Most mutations identified in KRAS are hot spot mutations in codon 12, 13 and 61 (8 over 11), consistent with published results. More importantly, we have identified 4 point mutations in exon 19 of PIK3CA which encodes the kinase's catalytic domain. They are K944N, F930S, V955G, and K966E. K944N was identified in 3 patients. We have also identified 3 point mutations in BRAF, one of which is the hot spot mutation V600E. Other sporadic mutations identified were in AKT1, EGFR, NRAS, and PTEN as shown in Supplemental Table 5 and 6.

### Correlation of Candidate Mutation Variant Frequency and Dynamic Level of CEA, CA 19-9, and CT Scan

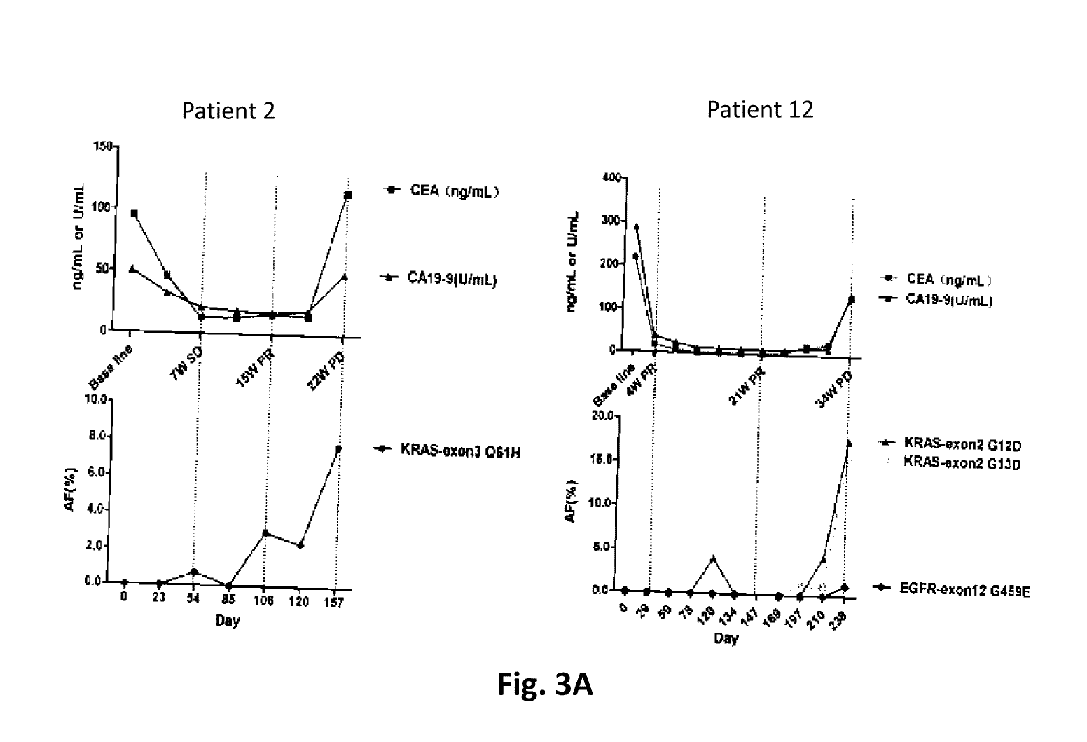

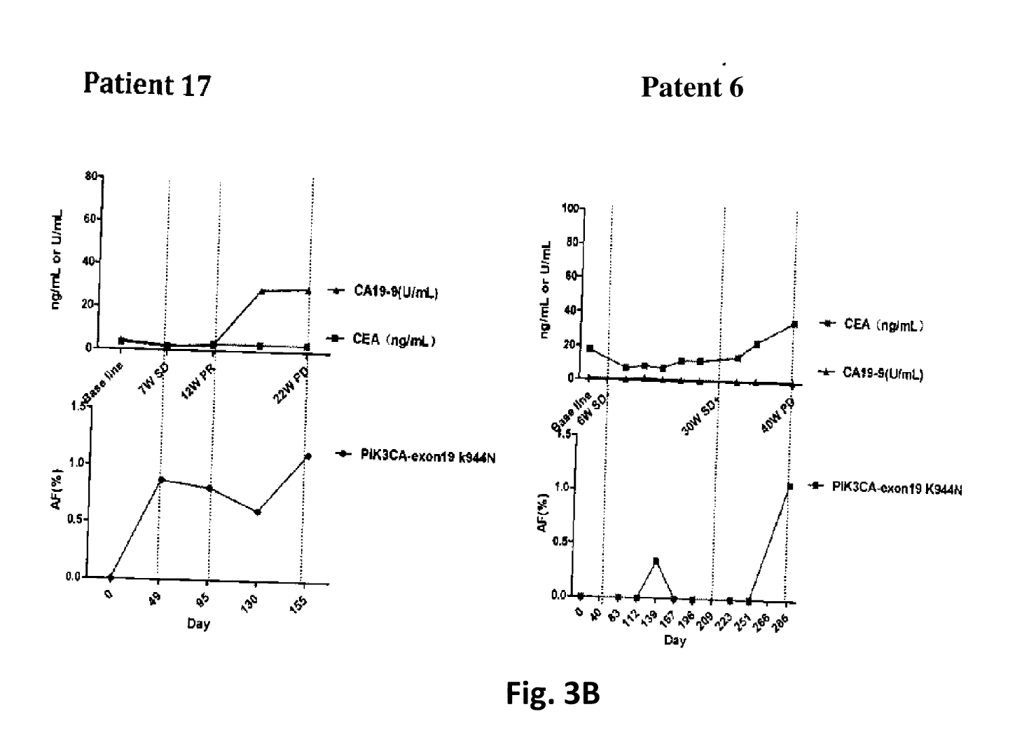

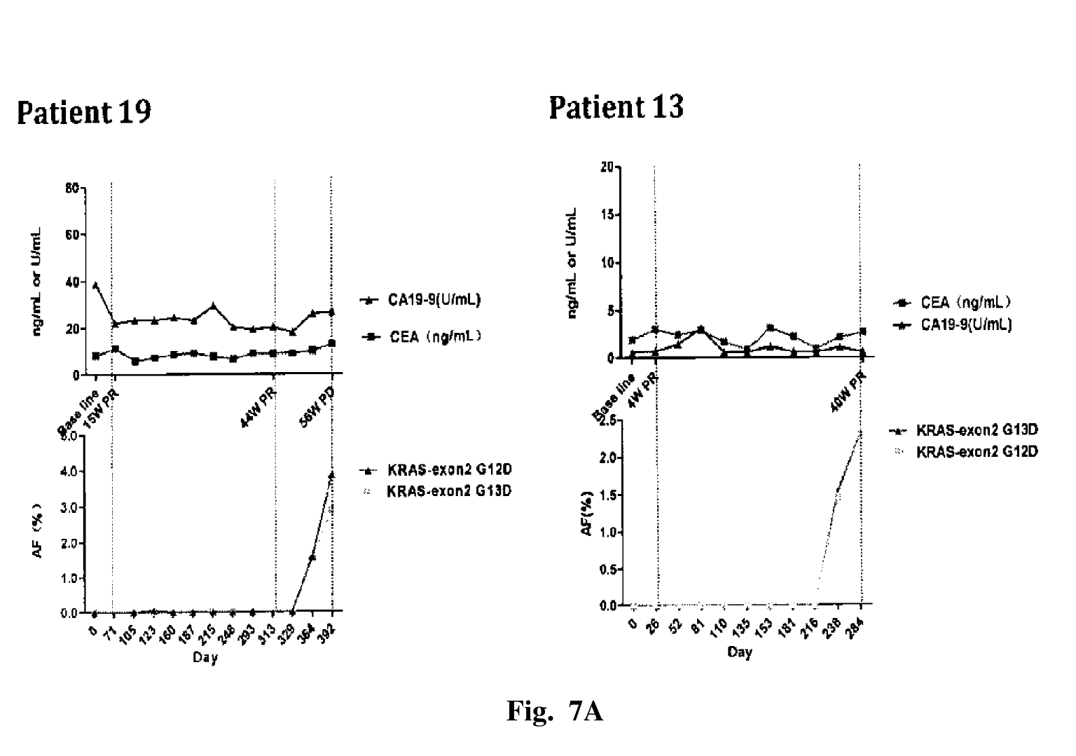

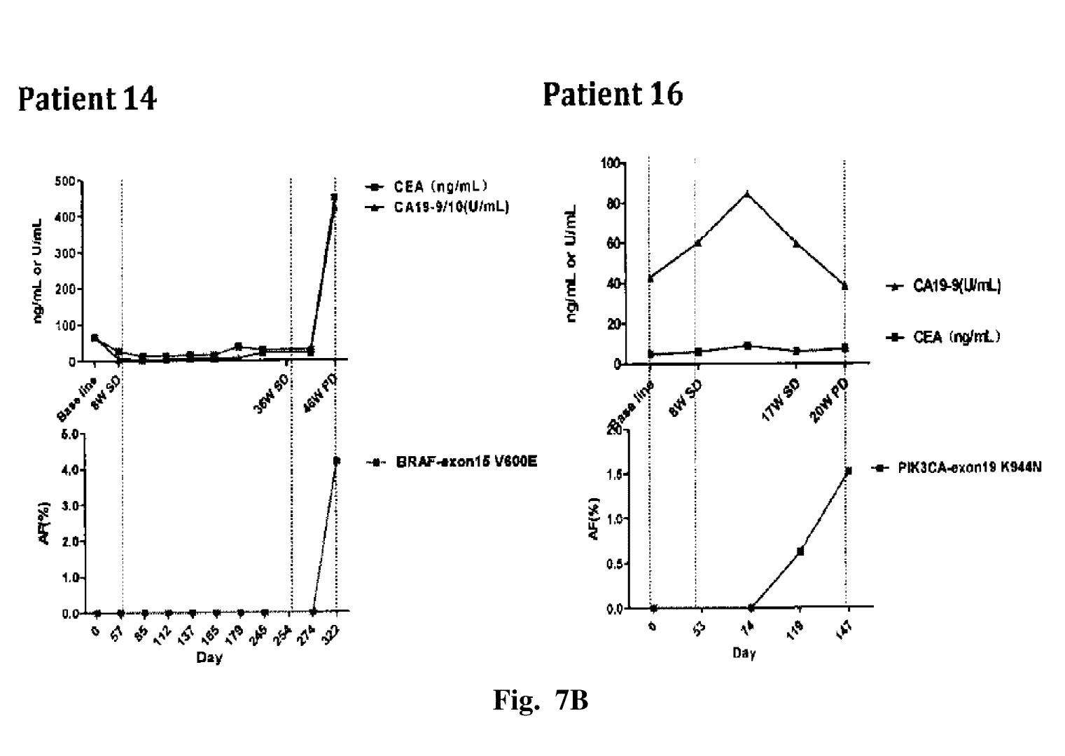

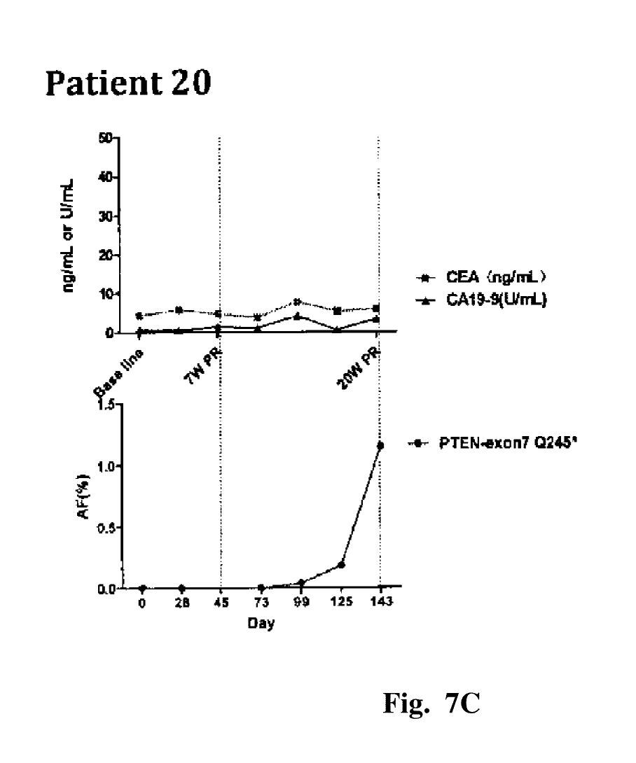

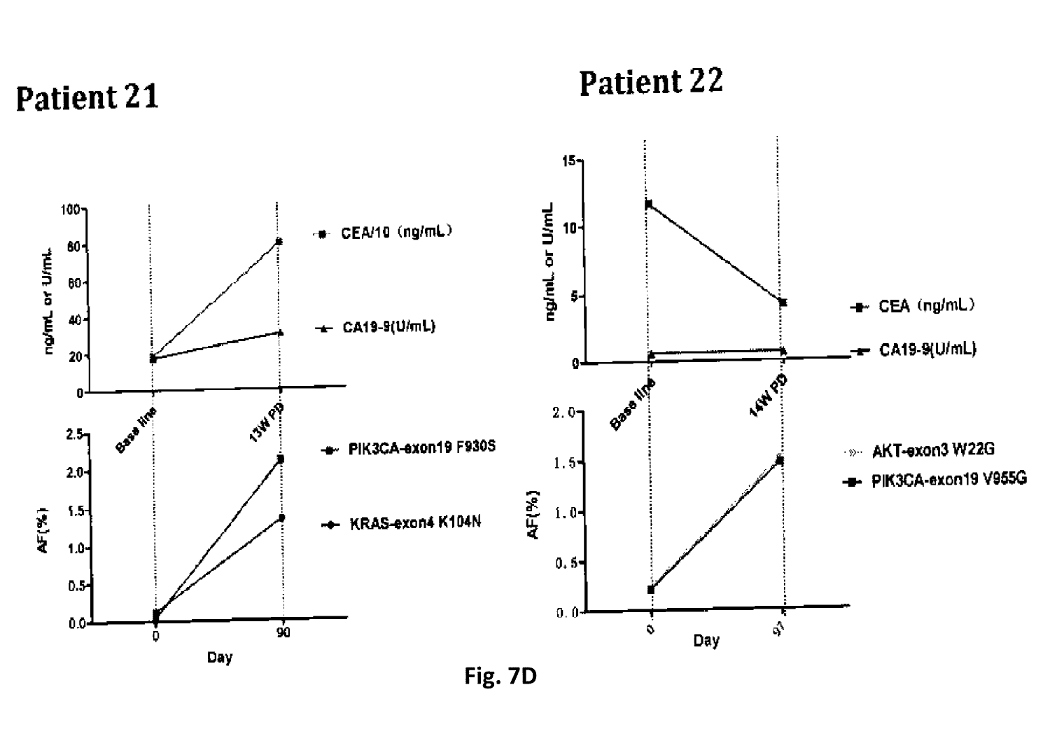

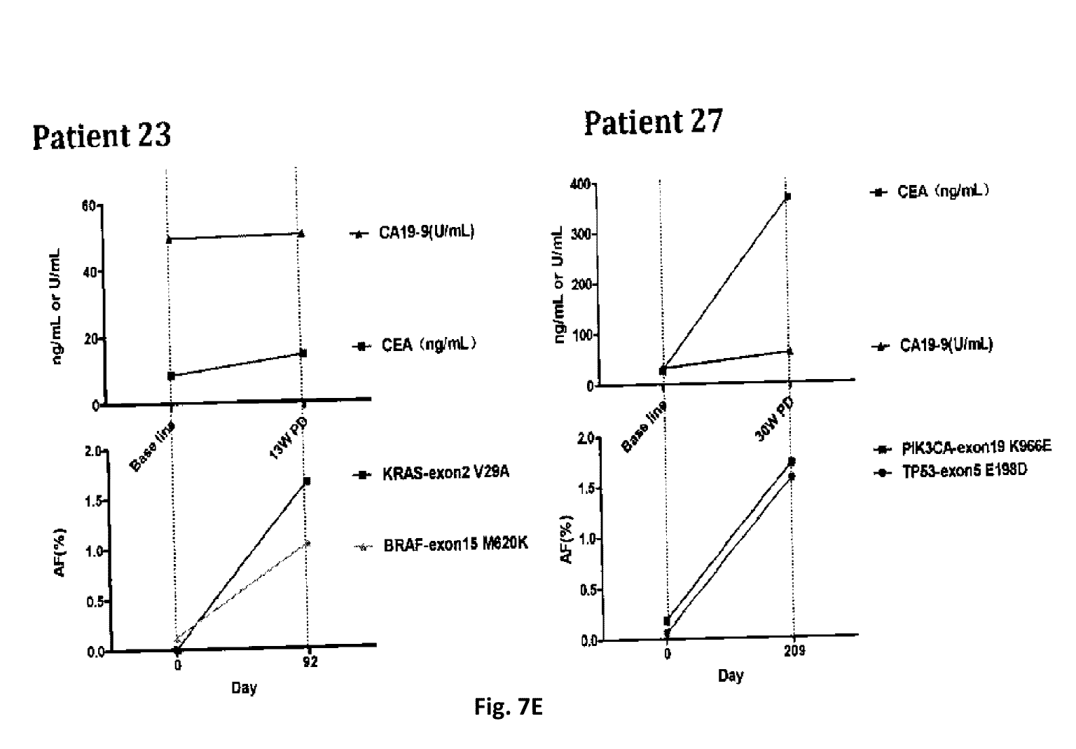

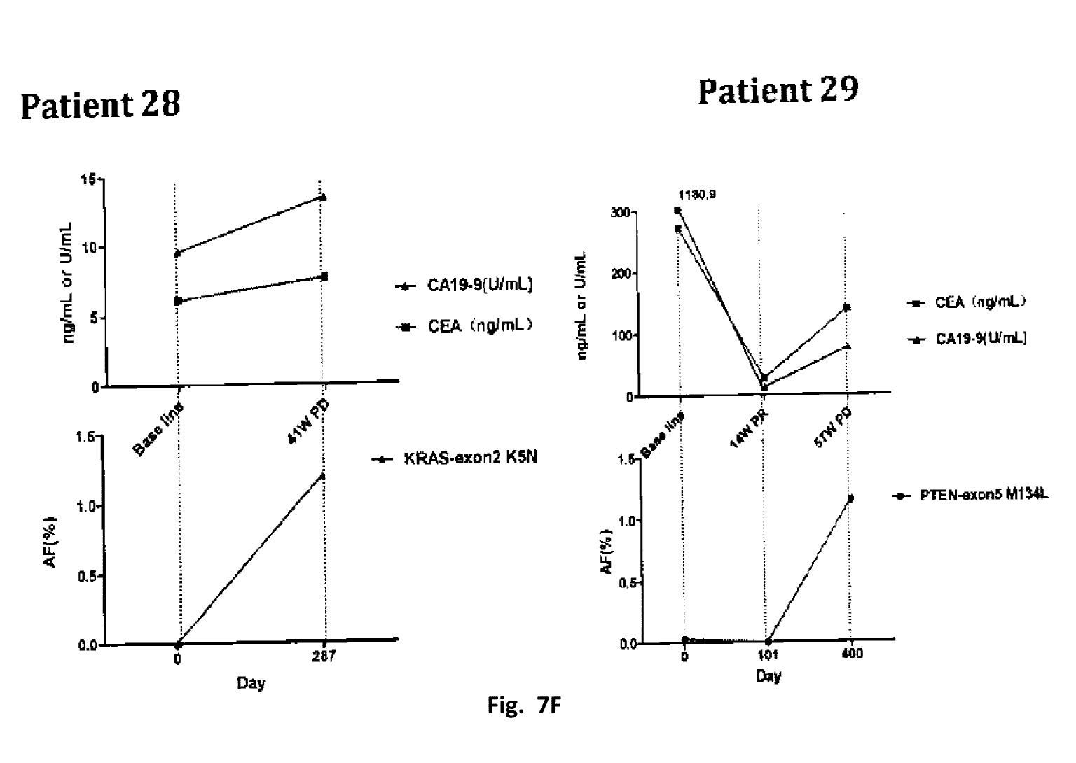

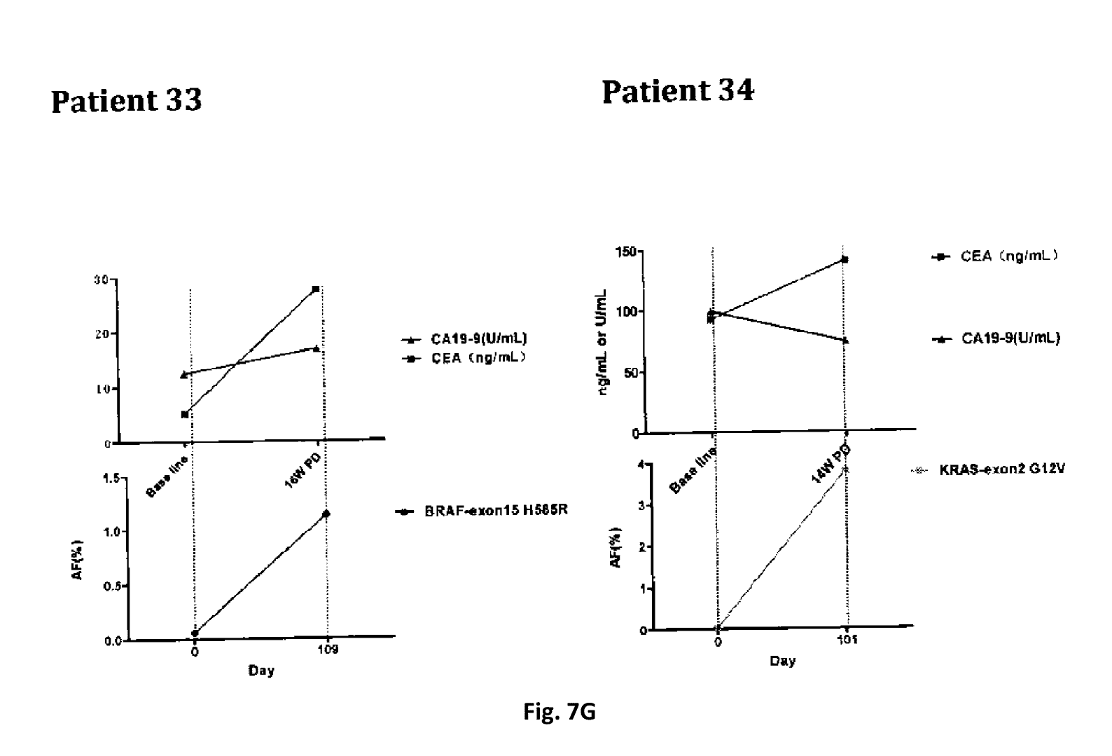

Candidate mutations were identified in longitudinal plasma samples from 9 out of 20 patients with acquired cetuximab resistance. We compared changes in variant frequency of those identified mutations in each patient with corresponding serum tumor biomarker and imaging results (FIGS. 3A-3B, FIG. 7). KRAS mutations were identified in 4 patients (2, 12, 13 and 19). Patient 2 had Q61H mutations. Patient 12 had multiple KRAS mutations (G12D/G13D) along with EGFR G459E. Patient 13 and 19 both had G12D and G13D double mutations. PIK3CA mutations were identified in 3 patients (6, 16, 17). Only 2 out of the 9 patients had mutations in other genes. Patient 14 had BRAF V600E mutation, and patient 20 had PTEN Q245* mutation. AKT1 W22G mutation was observed in patient 22. All those mutations described above exhibited increasing variant frequencies during treatment. The levels of candidate cetuximab resistant mutations went up prior to progression as determined by imaging. An average of 10 weeks of lead time (4.0-18.1 weeks) were observed.

Previous studies reported that drug resistant mutations could be detected in plasma 4-5 months prior to progression as determined clinically. We showed a shorter lead time here mainly because we used a different screening strategy. We selected mutations present only at PD, but not before treatment. Those mutations tend to be low in frequency and cannot be detected at an earlier time point. Other factors may also affect the lead time including detection sensitivity, different gene mutations contribute different power to drug resistance, presence of companying mutations besides the mutation quantitated, whether the mutation quantitated preexisted before therapy was initiated, etc.

### Correlation of Mutations in KRAS, BRAF, PIK3CA and Cetuximab Resistance

We further analyzed hot spot mutations in KRAS, BRAF, and mutations we identified in PIK3CA to better understand their effects on acquired cetuximab resistance. We selected 21 patients who had one or more of the following mutations (KRAS codon 12, 13, 61, BRAF 599, 600 and the PIK3CA mutations we identified) in tumor tissue or plasma (Table 1). In 13 out of 21 cases, we detected KRAS, BRAF, or PIK3CA mutations in tumor tissue before therapy, despite the fact KRAS and BRAF were tested to be wild type by Sanger Sequencing before cetuximab therapy. Among those, 4 exhibited novel mutations in plasma circulating DNA. Patients 16 and 17 with KRAS mutation in tumor tissue showed PIK3CA K944N mutation in plasma upon PD. Patient 22 with BRAF T599I and V600E double mutations in tumor developed PIK3CA V955G mutation upon PD. Patient 34 had PIK3CA L938* mutation in metastatic tumor tissue, who later was detected to have KRAS G12V hot spot mutation in ctDNA. Besides KRAS mutations as reported previously, our results indicate PIK3CA and BRAF mutations may also play important roles in acquired cetuximab resistance. The remaining 8 cases had no detectable mutations in these three genes. We detected KRAS mutations in four cases (patients 2, 12, 13, 19), including double KRAS mutations in 3 cases (patients 12, 13, 19). 3 cases harbored PIK3CA mutations (K944N, F930S, K966E) and one had BRAF V600E mutations. This further support the correlation between PIK3CA/BRAF mutations with cetuximab acquired resistance.

Prognostic Value of BRAF Mutations in Tumor Tissue, Mutations Detected in ctDNA Before Cetuximab Treatment, and Presence of Residual Primary Tumor

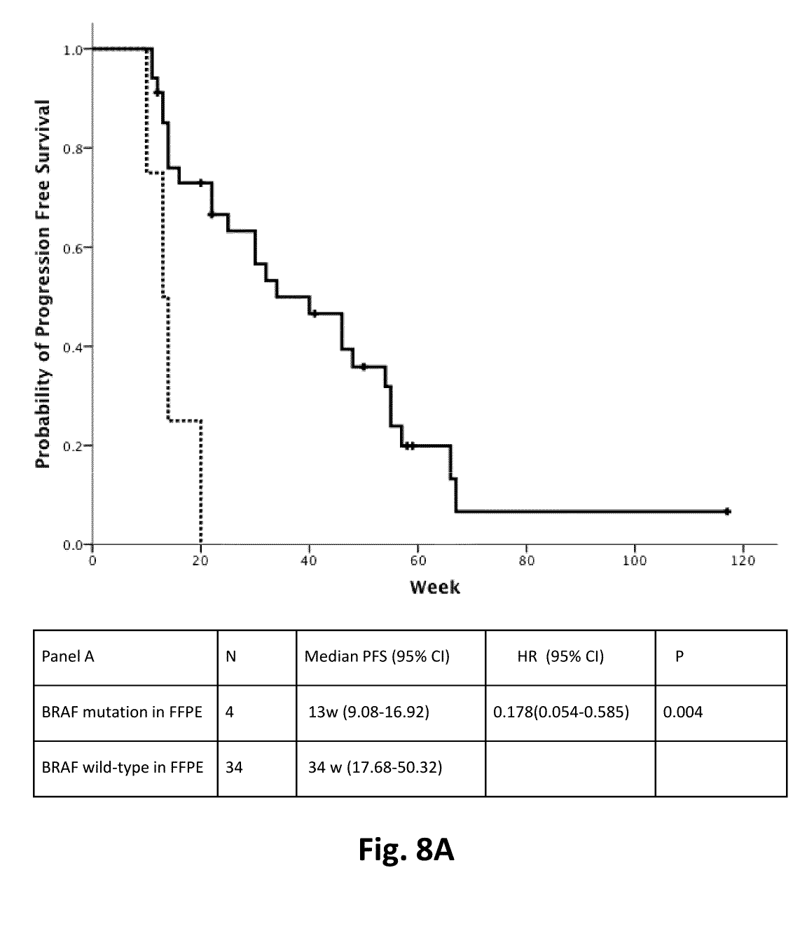

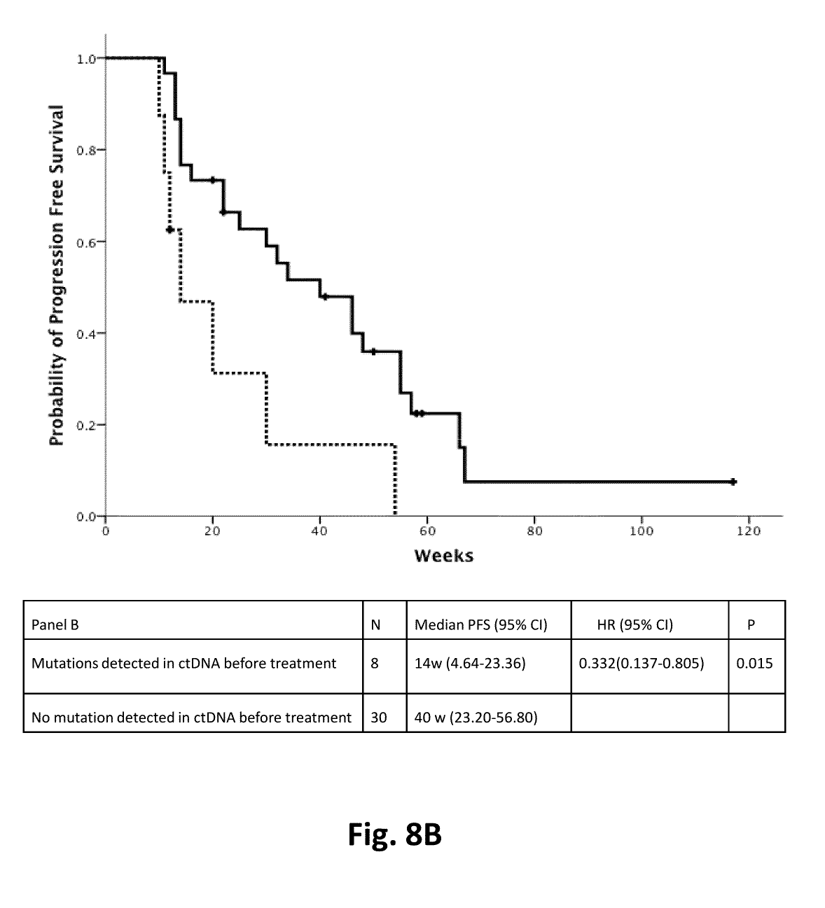

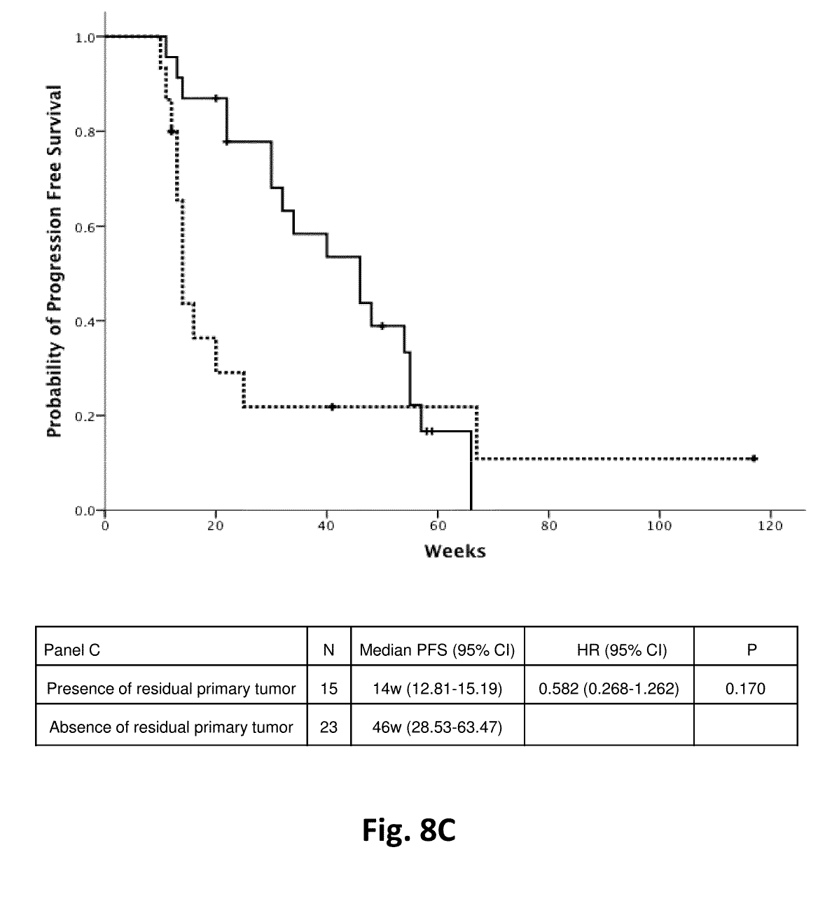

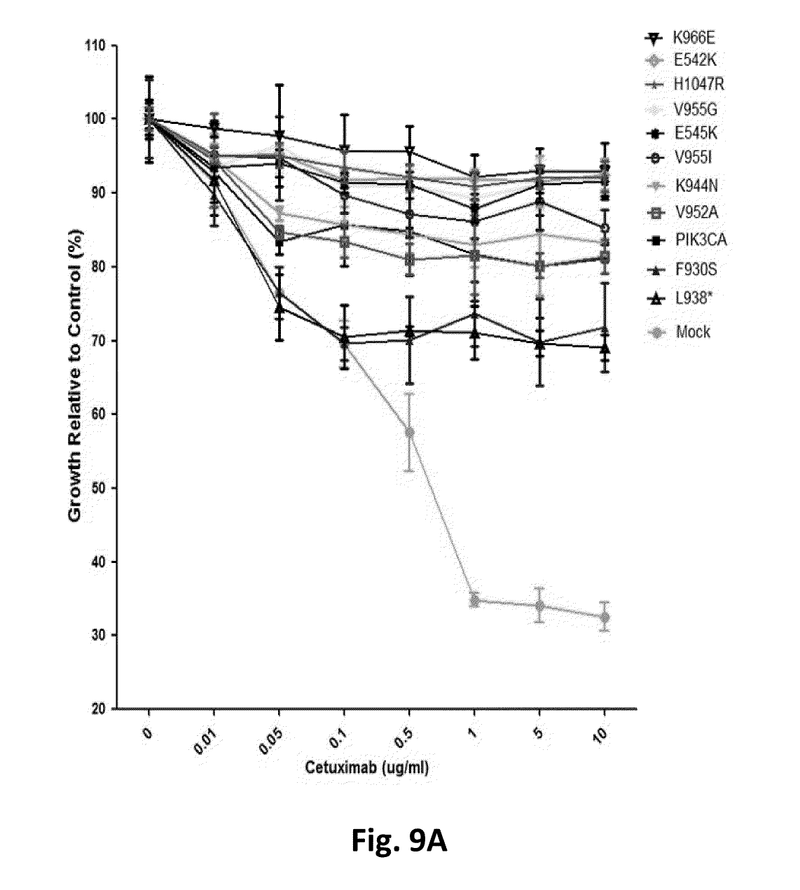

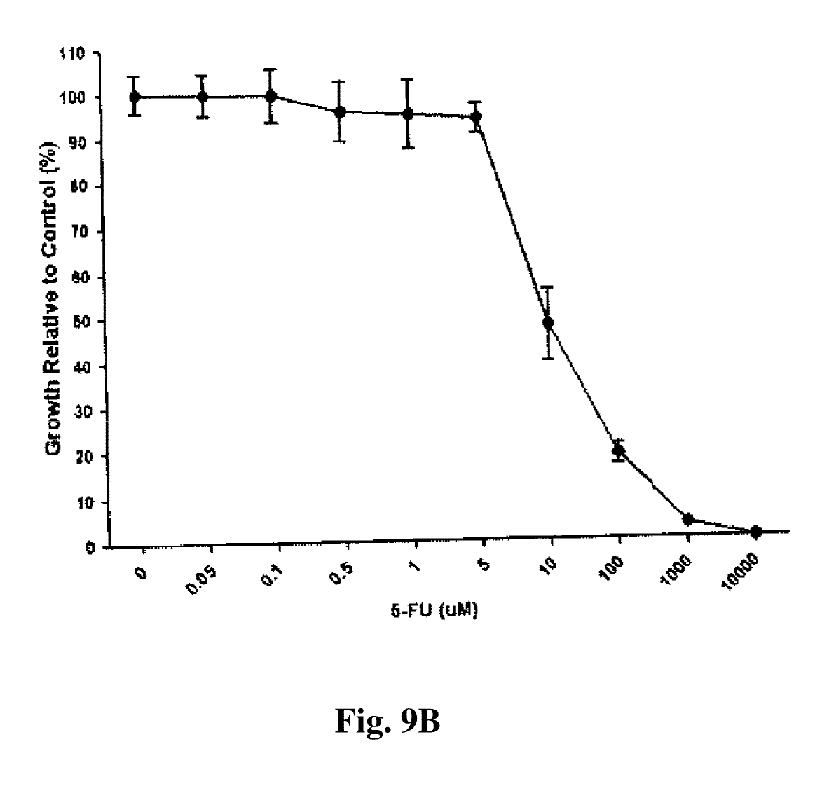

Together with clinical findings, we found that patients with BRAF mutations in tumor tissue (patients 22, 35, 36, 44, 50), any mutations detected in ctDNA before treatment (patient 3, 35, 37, 42, 43), or the presence of residual primary tumor had poor clinical outcome as compared with those without any of the above situations. In order to confirm our findings, we further compared the differences of PFS in all 38 acquired resistance patients grouped by above three situations. Our results demonstrated that patients with BRAF mutations in FFPE, or with mutations detected in ctDNA before treatment showed significantly shorter PFS as compared with those with wild type BRAF (Harzad Ratio 5.611, 95% confidence interval, 1.71 to 18.42; P=0.004) or without any mutation in ctDNA (Harzad Ratio 0.332, 95% confidence interval, 0.137 to 0.805; P=0.015), respectively; Patients with the presence of residual primary tumor had a meaningful trends towards shorter PFS as compared with those with the absence of residual primary tumor (Harzad Ratio 0.582, 95% confidence interval, 0.268 to 1.262; P=0.170) (FIG. 8).

### Pre-Existence of Resistant Mutations in Tumor Tissues as Indicator for Primary Vs Acquired Drug Resistance

We also looked for pre-existing mutations in the same panel of genes related to EGFR targeted therapy in participating patients. We analyzed 50 patients and identified 62 different somatic mutations in formalin fixed paraffin embedded (FFPE) tumor samples from 30 patients as shown in FIGS. 2A-2D. Majority of tumor tissues collected in this study were primary tumor tissues from surgery, median 9.3 months (0.4-58.6 months) to diagnosis of metastatic disease. In the 8 primary cetuximab resistant patients, the numbers of somatic mutations were uniformly distributed (17.4% in KRAS, NRAS, EGFR, and PTEN each), while in the 22 acquired cetuximab resistant patients, somatic mutations clustered in KRAS and TP53 at 30.8% each. The number of mutations in other genes tends to be low (FIG. 2a). Those results suggesting that different mutation profiles in genes downstream of EGFR signal transduction pathways may be informative to predict primary vs. acquired resistance during cetuximab treatment.

Among the 62 somatic mutations in FFPE tumor samples, only 14 of which were detected in plasma before treatment. Detection rate of mutations is 21% ( 8/38) and 50% ( 6/12) in acquired resistance and primary resistance patients, respectively (FIG. 2b). Furthermore, we found whether those mutations in FFPE samples can be detected in plasma did not correlate with their variant frequencies (Supplementary Table 2). Those results indicated the following: First, tumor would evolve by itself and during treatments. While majority of the FFPE tumor samples analyzed were from primary tumor, mutations profiles would change between removal of primary tumor and before centuximab treatment. Second, the fact a higher percentage of mutations from primary tumor tissues were detected in primary resistance patients, suggesting acquired resistance rely more on lower frequency or emerging mutations. Thus, mutations detected in circulating DNA from plasma before treatment, rather than those in primary tumors better represent mutational background before cetuximab treatment. If the purpose of the analyses is to identify emerging mutations that correlate with resistance during the treatments, our rational to focus on longitudinal plasma samples would allow us to more effectively and accurately identify mutation correlate with acquired resistance.

### Functional Analysis of PIK3CA Mutations

PIK3CA encodes the catalytic subunit of PI3K. It interacts with regulatory domain p85 to convert substrate PIP2 (phosphatidyli-nositol (4, 5)-bisphosphate) to PIP3 (phosphatidyli-nositol (3, 4, 5)-trisphosphate). PIP3 can interact with the N terminal PH domain of Protein Kinase B (PKB, AKT), activate AKT signal transduction pathway, which plays a very important role in the tumorgenesis and development of CRC, prostate cancer, breast cancer, ovarian cancer, hepatocellular carcinoma, lung cancer and melanoma.

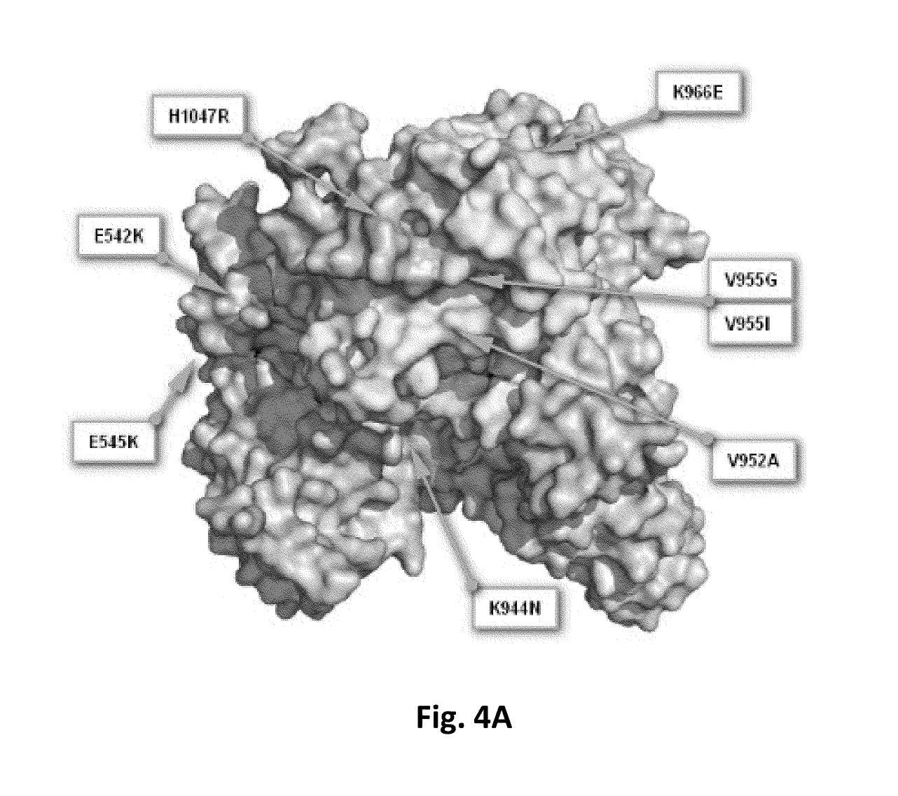

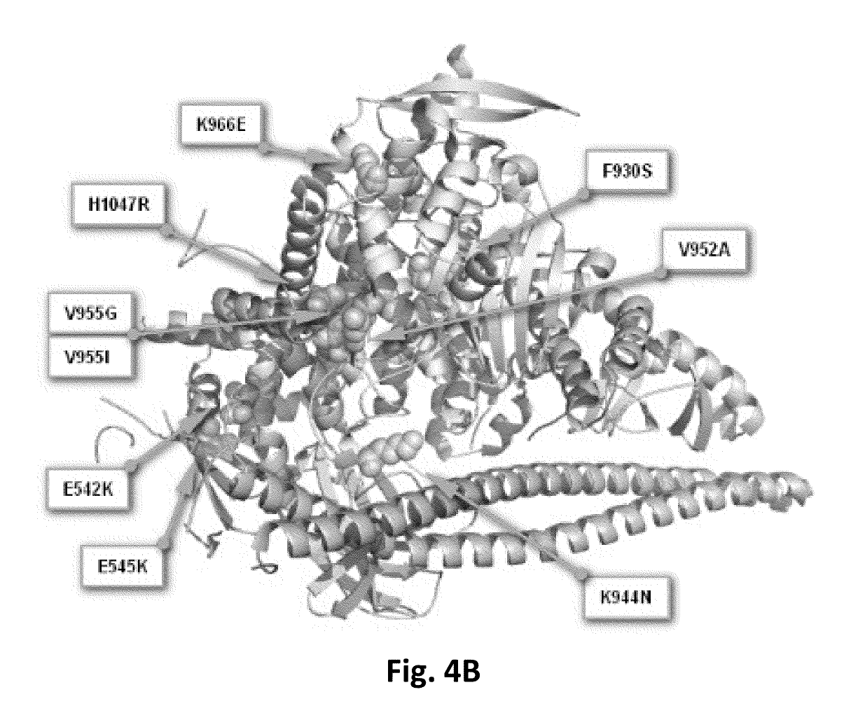

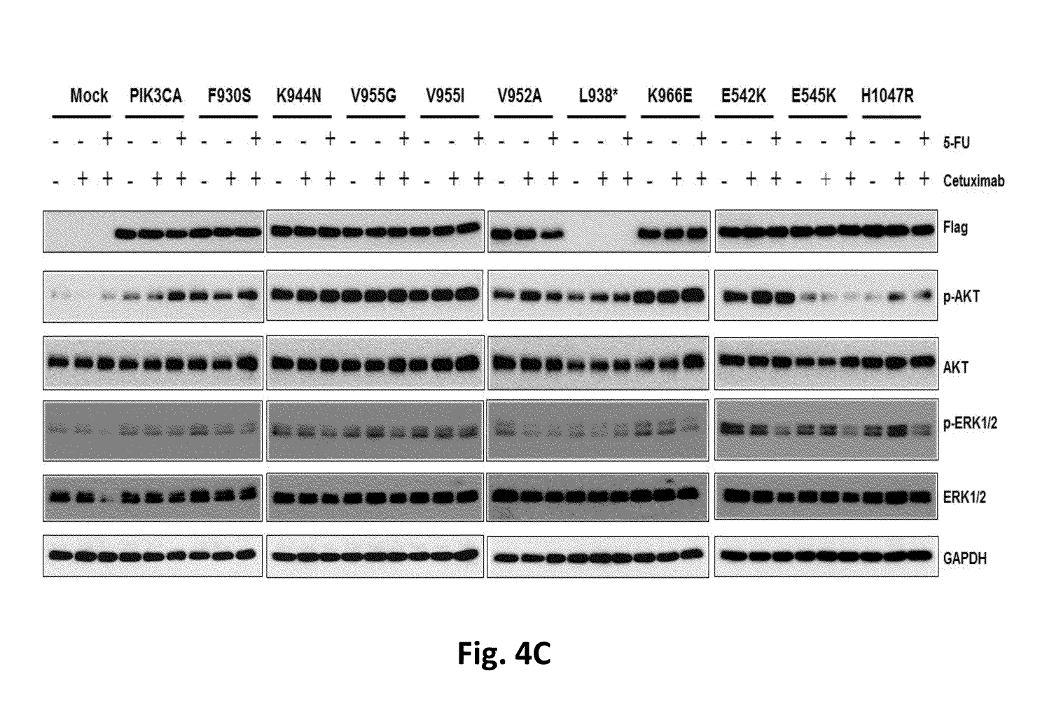

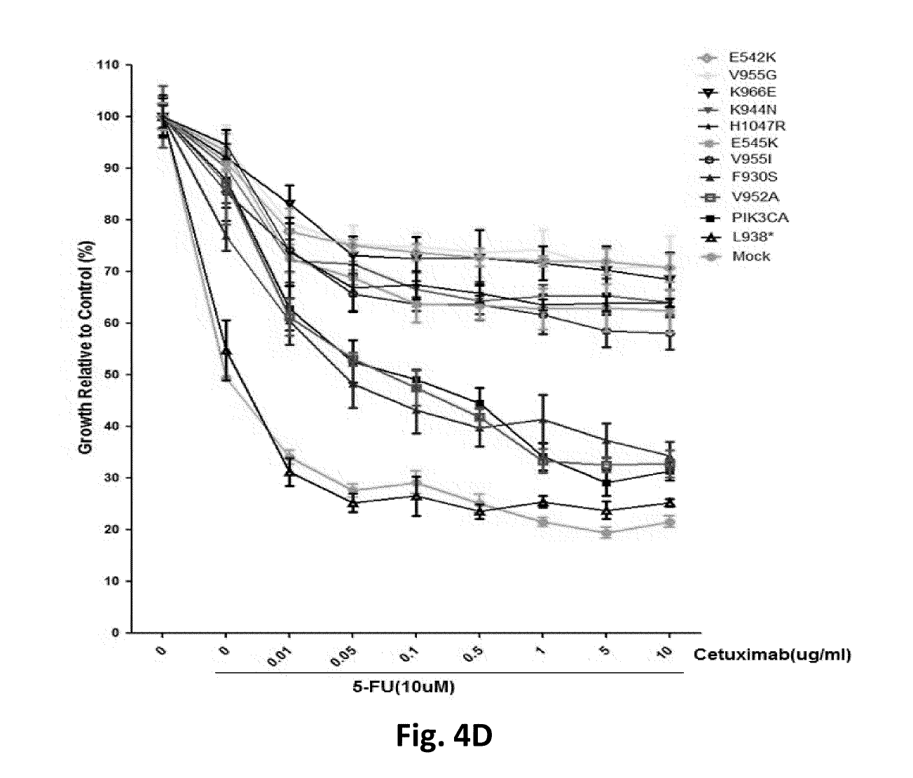

Protein structural functional analysis suggested: F930S abolished π interaction between F930 and Y836; K944N abolished ionic bond interaction with D464 while may form hydrogen bond interaction with K453, E457 and R577 in the regulatory subunit a; V955G, V955I and V952A changed the size of hydrophobic side chains at critical protein functional areas; K966E changed an attractive interaction to repellent interaction in ionic bond formation. All those mutations would affect the structural configuration of PIK3CA, resulting in functional changes of the protein. (FIG. 4a).

We included all PIK3CA mutations in this functional analysis, consist of 6 predicted “damaging” by both PolyPhen-2 and SIFT (K944N, F930S, V955G, K966E, V952A, L938*), and one (V955I) that was predicted as “damaging” by PolyPhen-2 only. V955 is located in the surface area of PIK3CA based on protein structure analysis. Both V955G and V955I mutations should affect the hydrophobic side chains similarly, thus contributing equally to alterations of protein functions. Results supported V955I had similar functional effect to V955G and other PIK3CA mutations in terms of cetuximab resistance. Since we did not include V955I in the data presented (FIG. 2c, 2d, FIGS. 3A-3B), we looked into clinical information of the patient carrying this mutation. We found that this patient (Patient 7) carried KRAS G12S mutation in primary tumor sample and V955I in the ctDNA samples during therapy. The mutation frequencies increased from 0.11% to 1.21% during the process of therapy, suggesting it contributes to cetuximab resistance. In prognostic analysis, this patient did not carry residual primary tumor, with no BRAF mutation(s) and no mutations detected in plasma samples before therapy. Thus she enjoyed PFS as long as 46 weeks, consistent with our overall results and conclusions.

The next question we tried to address was whether candidate mutations, screened out from the crowd using the strategy described, are functional candidates that would contribute to resistance by affecting cell growth regulation. Two types of experiments were done to answer the question. We looked at growth curve of Difi cells transfected with both wild type PIK3CA and mutated PIK3CA carrying mutations we identified: K944N, F930S, V955G, V955I, K966E. V952A and L938* were both identified in FFPE tumor samples. We also included the V955I in our functional studies.

To study the functional properties encoded by the mutations we identified, we over expressed wild type and PIK3CA carrying mutations to be tested in Difi cells cultured in serum free medium. Previously reported PIK3CA mutations E542K, E545K and H1047R in exon 9 and 20 were also included in the test, serving as reference. Effects on AKT and ERK1/2 phosphorylation were used as readout to evaluate the function of various mutants. We observed that PIK3CA mutants K944N, V955G, V955I and K966E demonstrated the most significant effect in increasing the phosphorylation level of AKT and ERK1/2. Such level of activation was not affected by the addition of cetuximab alone or in combination with 5-FU. Other PIK3CA mutations F930S, V952A, buried internally in the protein, as well as truncation mutation L938* had less significant effect on AKT and ERK1/2 phosphorylation, especially in the presence of cetuximab and/or 5-FU (FIG. 4b).

We also looked at cell growth in the presence of different concentration of cetuximab, in combination with 10 μM 5-FU in serum free medium. Compared to mock transfected cells, Difi cells overexpressing K944N, V955G, V955I, K966E, E542K, E545K and H1047R exhibited the most resistance. Wild type PIK3CA, mutants F930S, V952A exhibited moderate resistant to drug treatment, and truncation mutation L938* did not show any resistance (FIG. 4c). All experiments suggested candidate mutations (K944N, V955G, V955I and K966E) we selected are functional.

### DISCUSSION

Recent publications have demonstrated the use of highly sensitive detection methods to identify tumor specific mutations in plasma circulating DNA, promoting the concept of using “liquid biopsy” to monitor acquired drug resistance. Two successful strategies were used: One is based on known drug resistant mutations. Researchers would follow the level of those known mutations in circulating DNA, to assess the likelihood of resistance. For example, Diaz et al reported that monitoring certain KRAS mutations could predict resistance to panitumumab in mCRC patients even before progression is determined clinically. Another approach is to start with exom sequencing on tumor biopsies, to identify candidate mutations contributing to resistance, followed by digital PCR or other quantitative methods to monitor the level of those mutations during disease progression in longitudinal plasma samples. Both strategies monitored a list of mutations based on reported “hotspot” mutations or “common” mutations identified within the group of patients participating the study. If patients with acquired resistance have mutations in genes beyond the scope of monitoring, researchers will not be able to identify them.

To achieve a more personalized and comprehensive understanding of resistance to EGFR targeted therapy, we did two things differently. First, we used amplicon ultra deep sequencing technology to directly monitor mutations in 18 exons from 8 genes on every plasma samples to understand resistance at individual patient's level. Second, we focused on mutations absent in tumor biopsies due to their low variant frequency and mutations did not exist before therapy. That should lead us to not only discoveries of novel mutations correlating to acquired resistance, but also an understanding of resistance for each patients even when they have diverse mutation profiles.

The technical challenge of this study came from two aspects: the fact that only a fraction of cell free circulating DNA was derived from tumor and the difficulty to reliably identify SNVs with low variant frequency in amplicon sequencing. Both aspects prompted us to conceive an innovative strategy to screen for drug resistant mutations in the crowd of random, low frequency variants.

We selected a panel of 8 genes involved in two major EGFR cell signal transduction pathways. Based on our screening strategy, we identified mutations not detectable in FFPE tumor biopsies or in plasma samples before therapy in the same patient. Those low frequency mutations would have been missed if we used routine bioinformatic tools with filtering criteria based on sequencing depth, variant frequency, etc. We identified novel mutations in PIK3CA, AKT and other genes. In the case of PIK3CA, different mutations were found in different patients; however, they were demonstrated to have similar contribution to cetuximab resistance in both cell based assays and protein structural function prediction. Thus, we obtained through this study, a more personalized and comprehensive list of mutations correlating with acquired resistance to cetuximab.

Comparing mutation profiles of before therapy and after progression, we observed that mutations tend to distribute across the panel of genes in plasma samples before therapy. Those mutations not only present in RAS/RAF/MEK/ERK pathway as well as TP53 gene, but also in PIK3CA/PTEN/AKT pathway. KRAS and PIK3CA harbored the most mutations within the gene areas we assayed. We postulate such mutations include ones involved in tumor development and response to various treatments before cetuximab. Previous studies have demonstrated gene mutations in the EGFR signal transduction pathways such as RAS, RAF and PIK3CA contribute to resistance to anti-EGFR antibody therapy. However, most researches reported KRAS mutations play an important role in EGFR targeted therapy while PIK3CA and other gene mutations are more considered passenger mutations for acquired drug resistance. In this study, we identified a list of PIK3CA mutations in 6 out of 38 patients with acquired resistance. Similar low frequency mutations were also detected in AKT and PTEN. We also detected KRAS mutations in 8 out of 38 patients with acquired resistance. Those KRAS mutations had higher variant frequencies compared to PIK3CA mutations based on our observation, suggesting higher detection sensitivity was achieved in our study based on deep sequencing of ctDNA.

Interestingly, the length of PFS does not seem to correlate with the burden of metastatic tumor before therapy, but affected by the presence of primary tumor and the presence in BRAF mutations in tumor tissue and/or plasma samples before therapy. This may due to RAS and TP53 being the main driver for tumorigenesis of sporadic CRC. It is possible that KRAS mutations were present in primary tumor. It was under the detection limit of Sanger sequencing, which was reported to be around 5-10%. The existence of primary tumor would deliver more ctDNA to the blood. Wild type KRAS clones in primary tumor would be eliminated during therapy, remainder clones with KRAS mutations would expand and display resistance in relatively short period of time, resulting in shorter PFS. In patients with no residual primary tumor, a higher percentage of patients had PIK3CA mutations in addition to KRAS in the circulating DNA, suggesting complementary effect of those two genes on acquired cetuximab resistance. KRAS wild type tumor, under the selection pressure of cetuximab, clones with KRAS and/or PIK3CA already present in tumor or similar mutations developed during therapy would grow and expand, resulting in progression observed in medical imaging.

Many researches demonstrated highly sensitive method such as BEAMing can detect KRAS mutation in ctDNA, thus could be used to predict acquired resistance to EGFR targeted therapy. Contradictory results have been presented in various publications about whether PIK3CA mutations at exon 9 and 20 identified in tumor tissue correlate with drug resistance. Based on our understanding of cancer biology and tumor evolution, we selected mutations present only in plasma samples after progression, but not in primary tumor or plasma samples before therapy. Those are most likely to be mutations not reported previously. For mutations identified, we used two software (PolyPhen-2 and SIFT) to analyze the potential effect of those mutations on protein functions. Only mutations determined to be deleterious by both software are considered candidates for acquired resistance to cetuximab. Consistent with previous study, 46% of the mutations we identified are in KRAS. PIK3CA harbored 24% of the candidate mutations identified in this study, only next to KRAS. All point mutations identified in PIK3CA clustered in its catalytic kinase domain.

We included 7 PIK3CA mutations (5 identified in post PD plasma, 2 in FFPE tumor biopsies) in our functional analysis. All of them demonstrated cetuximab resistance. Both protein structural functional prediction and in vitro studies consistently clustered the mutations into two groups. The mutations present at the protein surface (K944N, V955G, V955I and K966E) had stronger effects increasing AKT and ERK1/2 phosphorylation in comparison to mutations present inside the protein. Those mutations also demonstrated stronger resistance to cetuximab combined with 5-FU than mutations located inside the protein. All PIK3CA mutations passed our selection, though low in variant frequency, were confirmed to be functional mutations related to cetuximab resistance. That result validated the effectiveness and accuracy of our screening strategy.

Many of the challenges in clinical studies involving acquired drug resistance lies in the difficulties of patients' willingness to provide multiple biopsies during the process and the level of representation of tumor biopsies in metastatic cancer patients with multiple solid tumors. Relying on the concept of “liquid biopsy” can circumvent those challenges. Using deep sequencing to cover as many genes in the pathway allowed us to take a more personal look in each patient for mutations causing acquired resistance to cetuximab. We identified candidate mutations correlate with drug resistance in 10 out 20 patients with longitudinal plasma samples. The remainder 10 may involve mutations outside the scope of our testing and/or other mechanisms such as transcription regulation or gene amplification. Recent studies indicate besides point mutations, copy number variations in KRAS, MET and ERBB2 also contribute to resistance to EGFR targeted therapy. Interestingly, patient 15 was detected to have CRC caused by FAP. Colorectomy was conducted to remove primary tumor. No relevant mutations were detected in neither primary tumor nor longitudinal plasma samples. The mechanism for acquired drug resistance may need to be investigated at a broader coverage of genes.

In summary, we have identified many novel mutations within the gene panel we selected involved in EGFR signaling pathway: PIK3CA in most cases, plus PTEN and AKT. Functional studies on multiple PIK3CA mutations we identified demonstrated all those mutations affected cell growth and level of phosphorylation in downstream targets. We believe acquired cetuximab resistant may result from various deleterious mutations in different genes involved in the EGFR cell signal transduction pathways. While previous studies have identified a few hot spot mutations in KRAS, the use of amplicon deep sequencing allowed us to understand acquired drug resistance at individual patient level. We are able to identify not only novel mutations correlate with progression as determined by imaging, but also detect those mutations an average of 10 weeks before progression. Moving forward, we should have a more comprehensive analysis of gene variations causing drug resistant to include more genes in the EGFR signaling pathway and take copy number variation (CNV) into consideration besides single nucleotide variation (SNVs). Improving the sensitivity of detection would also help us identify candidate mutations at earlier stage.

While the present invention has been described in some detail for purposes of clarity and understanding, one skilled in the art will appreciate that various changes in form and detail can be made without departing from the true scope of the invention. All figures, tables, appendices, patents, patent applications and publications, referred to above, are hereby incorporated by reference.

### REFERENCES

1. Koboldt, Daniel C and Zhang, Qunyuan and Larson, David. E and Shen, Dong and McLellan, Michael D and Lin, Ling and Miller, Christopher A and Mardis, Elaine R and Ding, Li and Wilson, Richard K (2012). “VarScan 2: somatic mutation and copy number alteration discovery in cancer by exome sequencing”. Genome research (Cold Spring Harbor Lab) 22(3): 568-576.
2. Larson, David E and Harris. Christopher C and Chen. Ken and Koboldt, Daniel C and Abbott, Travis E and Dooling, David J and Ley. Timothy J and Mardis, Elaine R and Wilson, Richard K and Ding, Li (2012). “SomaticSniper: identification of somatic point mutations in whole genome sequencing data”. Bioinformatics (Oxford Univ Press) 28 (3): 311-317.
3. Ding, Jiarui and Bashashati, Ali and Roth, Andrew and Oloumi, Arusha and Tse, Kane and Zeng, Thomas and Haffari. Gholamreza and Hirst, Martin and Marra, Marco A and Condon, Anne and others (2012). “Feature-based classifiers for somatic mutation detection in tumour—normal paired sequencing data”. Bioinformatics (Oxford Univ Press) 28 (2): 167-175.
4. Koboldt, D. C. et al. (2009). VarScan: variant detection in massively parallel sequencing of individual and pooled samples. Bioinformatics 25, 2283-2285.

What is claimed is:

1. A method of identifying a drug resistance-related mutation, comprising comparing variant frequencies of a single nucleotide mutation in genomic DNA from a patient before drug treatment and at different time points during the drug treatment, wherein the single nucleotide mutation with increased variant frequency is the drug resistance-related mutation.

2. The method of claim 1, wherein the variant frequency of the single nucleotide mutation is determined by amplicon ultra deep sequencing.

3. The method of claim 1, wherein the genomic DNA is extracted from cell free circulating DNA in plasma.

4. The method of claim 1, wherein the drug resistance-related mutation is only present in DNA samples after the drug treatment, and does not exist in the pre-treatment DNA samples.

5. The method of claim 1, wherein the drug resistance-related mutation has increasingly higher variant frequency during the course of drug treatment.

6. The method of claim 1, comprising the steps of:
a) calculating variant frequencies of single nucleotide mutations in DNA samples of a patient at pre-treatment and disease progression stages;
b) identifying single nucleotide mutations with significantly higher variant frequency in DNA samples at treatment/progressive disease (PD) stage than those in pretreatment DNA samples and making a treatment/PD-preferred mutation group;
c) selecting outlier mutations in the treatment/PD-preferred mutation group that have significantly higher variant frequencies at treatment/PD stage than those of the rest mutations in the same group, wherein the single nucleotide mutations with high variant frequency outliers are drug resistance-related mutations.

7. The method of claim 6, further comprising selecting single nucleotide mutations with variant frequency increasing in longitudinal samples during the course of drug treatment, wherein the selected mutations are the drug resistance-related mutations.

8. The method of claim 6, further comprising selecting single nucleotide mutations with loss or gain of function effect on a protein.

9. A method of predicting the occurrence of drug resistance, comprising the steps of:
a) calculating variant frequencies of single nucleotide mutations in DNA samples of a patient at pre-treatment stage and different time points during the course of drug treatment;
b) collecting a pool of mutations (treatment-preferred mutations) wherein the variant frequencies are significantly higher in the DNA samples of drug treatment stage than those in pretreatment samples;
c) detecting the occurrence of high frequency outlier mutations in the pool of treatment-preferred mutations, wherein the occurrence of the high frequency outlier mutation predicts the coming occurrence of drug resistance.

10. The method of claim 9, wherein the variant frequency is determined by amplicon ultra deep sequencing.

11. The method of claim 9, wherein the DNA sample is cell free circulating DNA in patient's blood sample.

12. The method of claim 9, wherein the treatment-preferred mutations are only present in DNA samples after treatment, but are absent in pretreatment samples.

13. The method of claim 9, wherein the drug resistance-related mutations cause gain or loss of function in a protein.
